(19) United States
(12) Patent Application Publication
(10) Pub. No.: US 2007/0254145 A1
(43) Pub. Date: Nov. 1, 2007

(54) Molded elements

(75) Inventors: Philip Andrew Sawin, Wyoming, OH (US); Astrid Annette Sheehan, Symmes Township, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/796,984

(22) Filed: Apr. 30, 2007

Related U.S. Application Data
(60) Provisional application No. 60/796,755, filed on May 1, 2006.
(60) Provisional application No. 60/880,599, filed on Jan. 16, 2007.

(51) Int. Cl. A61M 35/00; D04H 13/00; B32B 27/04; B32B 1/00

(52) U.S. Cl. 428/292.1; 428/297.4; 604/289; 428/187

(57) ABSTRACT

A molded fibrous structure comprising a molded element. The molded element may be hollow. The molded elements may provide for an increase in the fluid uptake of the fibrous structure. The molded element may provide a texture impression of a high level molded fibrous structure.

### CROSS REFERENCE TO RELATED APPLICATION

This application claims the benefit of U.S. Provisional Application No. 60/796,755 filed May 1, 2006 and U.S. Provisional Application No. 60/880,599, filed Jan. 16, 2007.

### FIELD OF THE INVENTION

A nonwoven fibrous structure comprising molded elements. The molded elements may improve and increase fluid uptake and retention. The molded elements may provide a highly molded texture impression to a user of the fibrous structure.

### BACKGROUND OF THE INVENTION

Historically, various types of nonwoven fibrous structures have been utilized as disposable substrates. The various types of nonwovens used may differ in visual and tactile properties, usually due to the particular production processes used in their manufacture. In all cases, however, consumers of disposable substrates suitable for use as wipes, such as baby wipes, demand strength, thickness, flexibility, texture and softness in addition to other functional attributes such as cleaning ability. Consumers often react to visual and tactile properties in their assessment of wipes.

Consumers often have a perception of the texture impression of a wipe based upon the appearance of the wipe itself, and, therefore, the perception is often subjective in nature. The texture of the wipe may provide visual signals to a consumer of product differentiation, strength, softness and cleaning efficacy. Additionally, wipes should have fluid uptake and retention properties such that they quickly acquire fluid during processing and remain wet during storage, and sufficient thickness, porosity, and texture to be effective in cleaning the soiled skin of a user.

The characteristics of strength, thickness, flexibility, fluid uptake and retention and texture impression may be affected by any hydromolding (also known as hydroembossing, hydraulic needlepunching, etc.) of the nonwoven fibrous structure during manufacture. Hydromolding is a means of introducing texture and/or design to the nonwoven structures. Various images and graphics may be hydromolded onto the nonwoven fibrous structure. The images and graphics may be a single image or graphic, a group of images or graphics, a repeating pattern of images or graphics, a continuous image or graphic or combinations thereof.

The fibrous web may be conveyed over a molding member, such as a drum, belt, etc., that may comprise a molding pattern of raised areas, lowered areas, or combinations thereof interspersed thereon. The pattern may be used to mold the image, graphic or texture onto the fibrous web thereby creating a molded fibrous structure. The resulting image, graphic, or texture on the fibrous structure may be a molded element of the fibrous structure.

Beyond providing a texture impression to a consumer, molding a fibrous structure may provide for an improvement in the fluid uptake performance of the nonwoven fibrous structure. Without being bound by theory, it is believed that fluid uptake of the fibrous structure may be a function of both the total fluid holding capacity (defined by capillary void space) and the ease with which the impinging liquid can enter these capillary void spaces. It is believed that hydromolding may create a disruption to the capillary nature of the void spaces. A highly molded fibrous structure may have a decrease in the amount of area that may contribute to the total effective capillary void space. This may, therefore, result in a reduction in the total fluid holding capacity. An unmolded fibrous structure may demonstrate a higher total fluid holding capacity due to a larger amount of capillary void space when compared with a highly molded fibrous structure. The capillary void space of an unmolded fibrous structure, however, may not be able to, funnel an impinging liquid throughout the fibrous structure as readily as a molded fibrous structure. There is, therefore, a need to optimize the amount of molding of a fibrous structure. There is a need to balance the properties of fluid uptake and retention of a molded fibrous structure. There remains a need to provide a substrate from such a molded fibrous structure.

Molding of a fibrous structure may also have an impact on the user's perception of a texture impression of the fibrous structure. Molded elements may be utilized on a fibrous structure to provide a user with a visual impression of the texture of the fibrous structure. It is surmised that the greater the number or size of molded elements, the greater the belief that the fibrous structure is soft to the touch and provides a better cleansing experience. High level molding of a fibrous structure may provide the user with an impression that the fibrous structure is highly textured. However, high level molding of the structure may have a negative impact on the benefit of fluid uptake of the structure, thereby resulting in a decrease in the performance of the structure.

Thus, there is a need to maintain the fluid uptake and retention properties of a fibrous structure and simultaneously maintain the texture impression of the fibrous structure. There remains a need to determine the level of molding that a fibrous structure may incorporate to maintain these properties of fluid uptake and retention and texture impression. There remains a need to provide a substrate from such a molded fibrous structure.

### SUMMARY OF THE INVENTION

The present invention relates to a fibrous structure comprising from about 5% to about 45% molded area. The fibrous structure may comprise at least one molded element. The fibrous structure may comprise synthetic fibers, natural fibers or combinations thereof.

The molded element may be a hollow element. The molded element may be selected from the group consisting of circles, squares, rectangles, ovals, ellipses, irregular circles, swirls, curly cues, cross hatches, pebbles, lined circles, linked irregular circles, half circles, wavy lines, bubble lines, puzzles, leaves, outlined leaves, plates, connected circles, changing curves, dots, honeycombs, and combinations thereof. The molded element may be selected from the group consisting of logos, indicia, trademarks, geometric patterns, surface images, and combinations thereof.

The fibrous structure may comprise at least two molded elements. One of the two molded elements may be smaller than the other molded element. The smaller molded element may comprise a radius unit. The smaller molded element may be within four radius units of the other molded element. The two molded elements may provide a high texture impression.

A substrate may comprise the fibrous structure. The substrate may comprise a composition.

### BRIEF DESCRIPTION OF THE DRAWINGS

FIG. 1 is a side view of a molding member of the present invention.

FIG. 2 is a top view of a molding member of the present invention shown with a fibrous web conveyed over the top of the molding member.

FIG. 3 is an illustration of a molding pattern of the present invention.

FIG. 4 is an illustration of a molding pattern of the present invention.

FIG. 5 is an illustration of a molding pattern of the present invention.

FIG. 6 is an illustration of a molding pattern of the present invention.

FIG. 7 is an illustration of a molding pattern of the present invention.

FIG. 8 is an illustration of a molding pattern of the present invention.

FIG. 9 is an illustration of a molding pattern of the present invention.

FIG. 10 is an illustration of a molding pattern of the present invention.

FIG. 11 is an illustration of a molding pattern of the present invention.

FIG. 12 is an illustration of a molding pattern of the present invention.

FIG. 13 is an illustration of a molding pattern of the present invention.

FIG. 14 is an illustration of a molding pattern of the present invention.

FIG. 15 is an illustration of a molding pattern of the present invention.

FIG. 16 is an illustration of a molding pattern of the present invention.

FIG. 17 is an illustration of a molding pattern of the present invention.

FIG. 18 is an illustration of a molding pattern of the present invention.

FIG. 19 is an illustration of a molding pattern of the present invention.

FIG. 20 is an illustration of a molding pattern of the present invention.

FIG. 21 is an illustration of a molding pattern of the present invention

FIG. 22 is an illustration of a molding pattern of the present invention.

FIG. 23 is an illustration of a molding pattern of the present invention.

FIG. 24 is an illustration of a molding pattern of the present invention.

FIG. 25 is an illustration of the fluid uptake of the molded area of a fibrous structure of the present invention.

FIG. 26 is an illustration of a molding pattern of a fibrous structure.

FIG. 27 is an illustration of a radius unit of the present invention.

### DETAILED DESCRIPTION OF THE INVENTION

“Air laying” refers herein to a process whereby air is used to separate, move, and randomly deposit fibers from a forming head to form a coherent, and largely isotropic fibrous web. Air laying equipment and processes are known in the art, and include Kroyer or Dan Web devices (suitable for wood pulp air laying, for example) and Rando Webber devices (suitable for staple fiber air laying, for example).

“Basis Weight” refers herein to the weight (measured in grams) of a unit area (typically measured in square meters) of the fibrous structure, which unit area is taken in the plane of the fibrous structure. The size and shape of the unit area from which the basis weight is measured is dependent upon the relative and absolute sizes and shapes of the regions having different basis weights.

“Carding” refers herein to a mechanical process whereby clumps of fibers are substantially separated into individual fibers and simultaneously made into a coherent fibrous web. Carding is typically carried out on a machine that utilizes opposed moving beds or surfaces of fine, angled, closely spaced teeth or wires or their equivalent to pull and tease the clumps apart. The teeth of the two opposing surfaces typically are inclined in opposite directions and move at different speeds relative to each other.

“Coforming” refers herein to include a spunmelt process, in which particulate matter, typically cellulose pulp, is entrained in the quenching air, so that the particulate matter becomes bound to the semi-molten fibers during the fiber formation process.

“Fibrous Structure” refers herein to an arrangement comprising a plurality of synthetic fibers, natural fibers, and combinations thereof. The synthetic and/or natural fibers may be layered, as known in the art, to form the fibrous structure. The fibrous structure may be a nonwoven. The fibrous structure may be formed from a fibrous web and may be a precursor to a substrate.

“gsm” refers herein to “grams per square meter.”

“Hollow” refers herein to a molded element in which the molded element defines a shape, such as a circle. The border of the molded element may be molded, but the interior of the molded element may be unmolded space and, therefore, hollow. The border of the molded element need not fully enclose the unmolded space, but may be concave relative to the interior unmolded space. The border of the molded element may be provided with gaps and may be considered a hollow element.

“Molded Element” refers herein to a texture, pattern, image, graphic and combinations thereof on a molded fibrous structure that have been imparted by hydromolding. The hydromolded texture, pattern, image, graphic and combinations thereof need not extend, without interruption, from a first edge of the molded fibrous structure to a second edge of the molded fibrous structure. The molded element may be a discrete element separate from another molded element. The molded element may overlap another molded element.

“Molding Member” refers to a structural element that can be used as a support for a fibrous web comprising a plurality of natural fibers, a plurality of synthetic fibers, and combinations thereof. The molding member may “mold” a desired geometry to the fibrous structure. The molding member may comprise a molding pattern that may have the ability to impart the pattern onto a fibrous web being conveyed thereon to produce a molded fibrous structure comprising a continuous molded element.

“Nonwoven” refers to a fibrous structure made from an assembly of continuous fibers, coextruded fibers, noncontinuous fibers and combinations thereof, without weaving or knitting, by processes such as spunbonding, carding, meltblowing, air laying, wet laying, coform, or other such processes known in the art for such purposes. The nonwoven structure may comprise one or more layers of such fibrous assemblies, wherein each layer may include continuous fibers, coextruded fibers, noncontinuous fibers and combinations thereof.

“Spunmelt” refers herein to processes including both spunlaying and meltblowing. Spunlaying is a process whereby fibers are extruded from a melt during the making of the coherent web. The fibers are formed by the extrusion of molten fiber material through fine capillary dies, and quenched, typically in air, prior to laying. In meltblowing, the airflow used during quenching is typically greater than in spunlaying and the resulting fibers are typically finer due to the drawing influence of the increased air flow.

“Substrate” refers herein to a piece of material, generally nonwoven material, used in cleaning or treating various surfaces, such as food, hard surfaces, inanimate objects, body parts, etc. For example, many currently available substrates may be intended for the cleansing of the perianal area after defecation. Other substrates may be available for the cleansing of the face or other body parts. A “substrate” may also be known as a “wipe” and both terms may be used interchangeably. Multiple substrates may be attached together by any suitable method to form a mitt.

“Texture impression” refers herein to the perceived visual impression by a user of a molded fibrous structure or substrate. The texture impression may be that of a low texture impression, a moderate texture impression or a high texture impression. The level of impression may be provided by the size and relative proximity of molded elements on the molded fibrous structure. A greater number of molded elements may provide a user with a high texture impression. A fewer number of molded elements which are large in size and spaced farther apart may also provide a user with a high texture impression. Small molded elements that are greater in number and closer together may provide a high texture impression. A fewer number of molded elements which are small in size and spaced farther apart may reduce the texture impression. Texture impression may provide a user a visual signal as to the softness and cleaning efficacy of the substrate.

Fibrous Web

The fibrous web can be formed in any conventional fashion and may be any nonwoven web which is suitable for use in a hydromolding process. The fibrous web may consist of any web, mat, or batt of loose fibers, such as might be produced by carding, air laying, spunlaying, and the like. The fibrous web may be a precursor to a nonwoven molded fibrous structure.

The fibers of the fibrous web, and subsequently the nonwoven molded fibrous structure, may be any natural, cellulosic, and/or synthetic material. Examples of natural fibers may include cellulosic natural fibers, such as fibers from hardwood sources, softwood sources, or other non-wood plants. The natural fibers may comprise cellulose, starch and combinations thereof. Nonlimiting examples of suitable cellulosic natural fibers include, but are not limited to, wood pulp, typical northern softwood Kraft, typical southern softwood Kraft, typical CTMP, typical deinked, corn pulp, acacia, eucalyptus, aspen, reed pulp, birch, maple, radiata pine, and combinations thereof. Other sources of natural fibers from plants include, but are not limited to, albardine, esparto, wheat, rice, corn, sugar cane, papyrus, jute, reed, sabia, raphia, bamboo, sidal, kenaf, abaca, sunn, cotton, hemp, flax, ramie, and combinations thereof. Yet other natural fibers may include fibers from other natural non-plant sources, such as, down, feathers, silk, and combinations thereof. The natural fibers may include extruded cellulose such as rayon (also known as viscose) and lyocell. The natural fibers may be treated or otherwise modified mechanically or chemically to provide desired characteristics or may be in a form that is generally similar to the form in which they can be found in nature. Mechanical and/or chemical manipulation of natural fibers does not exclude them from what are considered natural fibers with respect to the development described herein.

The synthetic fibers can be any material, such as, but not limited to, those selected from the group consisting of polyesters (e.g., polyethylene terephthalate), polyolefins, polypropylenes, polyethylenes, polyethers, polyamides, polyesteramides, polyvinylalcohols, polyhydroxyalkanoates, polysaccharides, and combinations thereof. Further, the synthetic fibers can be a single component (i.e., single synthetic material or mixture makes up entire fiber), bicomponent (i.e., the fiber is divided into regions, the regions including two or more different synthetic materials or mixtures thereof and may include coextruded fibers and core and sheath fibers) and combinations thereof. These bicomponent fibers can be used as a component fiber of the structure, they may be present to act as a binder for the other fibers present in the fibrous structure and/or they may be the only type of fiber present in the fibrous structure. Any or all of the synthetic fibers may be treated before, during, or after the process of the present invention to change any desired properties of the fibers. For example, in certain embodiments, it may be desirable to treat the synthetic fibers before or during processing to make them more hydrophilic, more wettable, etc.

In certain embodiments of the present invention, it may be desirable to have particular combinations of fibers to provide desired characteristics. For example, it may be desirable to have fibers of certain lengths, widths, coarseness or other characteristics combined in certain layers or separate from each other. The fibers may be of virtually any size and may have an average length from about 1 mm to about 60 mm. Average fiber length refers to the length of the individual fibers if straightened out. The fibers may have an average fiber width of greater than about 5 micrometers. The fibers may have an average fiber width of from about 5, 10, 15, 20 or 25 micrometers to about 30, 35, 40, 45 or 50 micrometers. The fibers may have a coarseness of greater than about 5 mg/100 m. The fibers may have a coarseness of from about 5 mg/100 m, 15 mg/100 m, 25 mg/100 m to about 50 mg/100 m, 60 mg/100 m or 75 mg/100 m.

The fibers may be circular in cross-section, dog bone shaped, delta (i.e., triangular cross-section), trilobal, ribbon, or other shapes typically produced as staple fibers. Likewise, the fibers can be conjugate fibers. The fibers may be crimped, and may have a finish, such as a lubricant, applied.

The fibrous web of the present invention may have a basis weight of between about 30, 40, 45, 50 or 55 gsm and about 60, 65, 70, 75, 80, 90 or 100 gsm. Fibrous webs for use in the present invention may be available from the J.W. Suominen Company of Finland, and sold under the FIBRELLA trade name. For example, FIBRELLA 3100 and FIBRELLA 3160 have been found to be useful as fibrous webs in the present invention. FIBRELLA 3100 is a 62 gsm nonwoven web comprising 50% 1.5 denier polypropylene fibers and 50% 1.5 denier viscose fibers. FIBRELLA 3160 is a 58 gsm nonwoven web comprising 60% 1.5 denier polypropylene fibers and 40% 1.5 denier viscose fibers. In both of these commercially available fibrous webs, the average fiber length is about 38 mm. Additional fibrous webs available from Suominen may include a 62 gsm nonwoven web comprising 60% polypropylene fibers and 40% viscose fibers; a fibrous web comprising a basis weight from about 50 or 55 to about 58 or 62 and comprising 60% polypropylene fibers and 40% viscose fibers; and a fibrous web comprising a basis weight from about 62 to about 70 or 75 gsm. The latter fibrous web may comprise 60% polypropylene fibers and 40% viscose fibers. The fibrous web of the present invention may be a 60 gsm nonwoven web comprising 40% pulp fibers and 60% lyocell fibers.

Molded Fibrous Structure

The fibrous web may be the precursor to a molded fibrous structure. The fibrous web may be conveyed over a molding member during or after manufacture. The molding member may comprise a molding pattern of raised areas, lowered areas, or combinations thereof interspersed thereon. Raised areas may also incorporate solid areas. Lowered areas may also incorporate void areas. The molding member may impart the pattern onto the fibrous web during a hydromolding process step thereby forming a fibrous structure comprising a molded element.

The molding pattern of raised and/or lowered areas may comprise images, graphics or combinations thereof and may comprise logos, indicia, trademarks, geometric patterns, images of the surfaces that a substrate (as discussed herein) is intended to clean (i.e., infant's body, face, etc.) or combinations thereof. They may be utilized in a random or alternating manner or they may be used in a consecutive, repeating manner. The images, graphics or combinations thereof may be a single image or graphic, a group of images or graphics, a repeating pattern of images or graphics, a continuous image or graphic, and combinations thereof.

The molded fibrous structure may comprise molded elements. The molded elements may be randomly arranged or may be in a repetitive pattern. The molded elements may comprise any image, graphic, texture, pattern or combinations thereof. The molded element may be any shape deemed suitable by one of ordinary skill. The molded element may be in the form of logos, indicia, trademarks, geometric patterns, images of the surfaces the fibrous structure is intended to clean (i.e., infant's body, face, etc.). The molded elements may be selected from the group consisting of circles, squares, rectangles, ovals, ellipses, irregular circles, swirls, curly cues, cross hatches, pebbles, lined circles, linked irregular circles, half circles, wavy lines, bubble lines, puzzles, leaves, outlined leaves, plates, connected circles, changing curves, dots, honeycombs, animal images such as paw prints, etc. and combinations thereof. The molded elements may be hollow elements. The molded elements may be connected to each other. The molded elements may overlap each other.

The fibrous structure of the present invention may take a number of different forms. The fibrous structure may comprise 100% synthetic fibers or may be a combination of synthetic fibers and natural fibers. In one embodiment of the present invention, the fibrous structure may include one or more layers of a plurality of synthetic fibers mixed with a plurality of natural fibers. The synthetic fiber/natural fiber mix may be relatively homogeneous in that the different fibers may be dispersed generally randomly throughout the layer. The fiber mix may be structured such that the synthetic fibers and natural fibers may be disposed generally nonrandomly. In one embodiment, the fibrous structure may include at least one layer comprising a plurality of natural fibers and at least one adjacent layer comprising a plurality of synthetic fibers. In another embodiment, the fibrous structure may include at least one layer that comprises a plurality of synthetic fibers homogeneously mixed with a plurality of natural fibers and at least one adjacent layer that comprises a plurality of natural fibers. In an alternate embodiment, the fibrous structure may include at least one layer that comprises a plurality of natural fibers and at least one adjacent layer that may comprise a mixture of a plurality of synthetic fibers and a plurality of natural fibers in which the synthetic fibers and/or natural fibers may be disposed generally nonrandomly. Further, one or more of the layers of mixed natural fibers and synthetic fibers may be subjected to manipulation during or after the formation of the fibrous structure to disperse the layer or layers of mixed synthetic and natural fibers in a predetermined pattern or other nonrandom pattern.

The fibrous structure may further comprise binder materials. The fibrous structure may comprise from about 0.01% to about 1%, 3%, or 5% by weight of a binder material selected from the group consisting of permanent wet strength resins, temporary wet strength resins, dry strength resins, retention aid resins and combinations thereof.

If permanent wet strength is desired, the binder materials may be selected from the group consisting of polyamide-epichlorohydrin, polyacrylamides, styrene-butadiene latexes, insolubilized polyvinyl alcohol, ureaformaldehyde, polyethyleneimine, chitosan polymers and combinations thereof.

If temporary wet strength is desired, the binder materials may be starch based. Starch based temporary wet strength resins may be selected from the group consisting of cationic dialdehyde starch based resin, dialdehyde starch and combinations thereof. The resin described in U.S. Pat. No. 4,981,557, issued Jan. 1, 1991 to Bjorkquist may also be used.

If dry strength is desired, the binder materials may be selected from the group consisting of polyacrylamide, starch, polyvinyl alcohol, guar or locust bean gums, polyacrylate latexes, carboxymethyl cellulose and combinations thereof.

A latex binder may also be utilized. Such a latex binder may have a glass transition temperature from about 0° C., −10° C., or −20° C. to about −40° C., −60° C., or −80° C. Examples of latex binders that may be used include polymers and copolymers of acrylate esters, referred to generally as acrylic polymers, vinyl acetate-ethylene copolymers, styrene-butadiene copolymers, vinyl chloride polymers, vinylidene chloride polymers, vinyl chloride-vinylidene chloride copolymers, acrylo-nitrile copolymers, acrylic-ethylene copolymers and combinations thereof. The water emulsions of these latex binders usually contain surfactants. These surfactants may be modified during drying and curing so that they become incapable of rewetting.

Methods of application of the binder materials may include aqueous emulsion, wet end addition, spraying and printing. At least an effective amount of binder may be applied to the fibrous structure. Between about 0.01% and about 1.0%, 3.0% or 5.0% may be retained on the fibrous structure, calculated on a dry fiber weight basis. The binder may be applied to the fibrous structure in an intermittent pattern generally covering less than about 50% of the surface area of the structure. The binder may also be applied to the fibrous structure in a pattern to generally cover greater than about 50% of the fibrous structure. The binder material may be disposed on the fibrous structure in a random distribution. Alternatively, the binder material may be disposed on the fibrous structure in a nonrandom repeating pattern.

Additional information relating to the fibrous structure may be found in U.S. Patent Application No. 2004/0154768, filed by Trokhan et al. and published Aug. 12, 2004, US Patent Application No. 2004/0157524, filed by Polat et al. and published Aug. 12, 2004, U.S. Pat. No. 4,588,457, issued to Crenshaw et al., May 13, 1986, U.S. Pat. No. 5,397,435, issued to Ostendorf et al., Mar. 14, 1995 and U.S. Pat. No. 5,405,501, issued to Phan et al., Apr. 11, 1995.

Substrate

The molded fibrous structure, as described above, may be utilized to form a substrate. The molded fibrous structure may continue to be processed in any method known to one of ordinary skill to convert the molded fibrous structure to a substrate comprising at least one molded element. This may include, but is not limited to, slitting, cutting, perforating, folding, stacking, interleaving, lotioning and combinations thereof.

The material from which a substrate is made should be strong enough to resist tearing during manufacture and normal use, yet still provide softness to the user's skin, such as a child's tender skin. Additionally, the material should be at least capable of retaining its form for the duration of the user's cleansing experience.

Substrates may be generally of sufficient dimension to allow for convenient handling. Typically, the substrate may be cut and/or folded to such dimensions as part of the manufacturing process. In some instances, the substrate may be cut into individual portions so as to provide separate wipes which are often stacked and interleaved in consumer packaging. In other embodiments, the substrates may be in a web form where the web has been slit and folded to a predetermined width and provided with means (e.g., perforations) to allow individual wipes to be separated from the web by a user. Suitably, the separate wipes may have a length between about 100 mm and about 250 mm and a width between about 140 mm and about 250 mm. In one embodiment, the separate wipe may be about 200 mm long and about 180 mm wide.

The material of the substrate may generally be soft and flexible, potentially having a structured surface to enhance its performance. It is also within the scope of the present invention that the substrate may include laminates of two or more materials. Commercially available laminates, or purposely built laminates would be within the scope of the present invention. The laminated materials may be joined or bonded together in any suitable fashion, such as, but not limited to, ultrasonic bonding, adhesive, glue, fusion bonding, heat bonding, thermal bonding, hydroentangling and combinations thereof. In another alternative embodiment of the present invention the substrate may be a laminate comprising one or more layers of nonwoven materials and one or more layers of film. Examples of such optional films, include, but are not limited to, polyolefin films, such as, polyethylene film. An illustrative, but nonlimiting example of a nonwoven sheet member which is a laminate of a 16 gsm nonwoven polypropylene and a 0.8 mm 20 gsm polyethylene film.

The substrate materials may also be treated to improve the softness and texture thereof. The substrate may be subjected to various treatments, such as, but not limited to, physical treatment, such as ring rolling, as described in U.S. Pat. No. 5,143,679; structural elongation, as described in U.S. Pat. No. 5,518,801; consolidation, as described in U.S. Pat. Nos. 5,914,084, 6,114,263, 6,129,801 and 6,383,431; stretch aperturing, as described in U.S. Pat. Nos. 5,628,097, 5,658,639 and 5,916,661; differential elongation, as described in WO Publication No. 2003/0028165A1; and other solid state formation technologies as described in U.S. Publication No. 2004/0131820A1 and U.S. Publication No. 2004/0265534A1, zone activation, and the like; chemical treatment, such as, but not limited to, rendering part or all of the substrate hydrophobic, and/or hydrophilic, and the like; thermal treatment, such as, but not limited to, softening of fibers by heating, thermal bonding and the like; and combinations thereof.

The substrate may have a basis weight of at least about 30 grams/m². The substrate may have a basis weight of at least about 40 grams/m². In one embodiment, the substrate may have a basis weight of at least about 45 grams/m². In another embodiment, the substrate basis weight may be less than about 100 grams/m². In another embodiment, substrates may have a basis weight between about 30 grams/m² and about 100 grams/m², and in yet another embodiment a basis weight between about 40 grams/m² and about 90 grams/m². The substrate may have a basis weight between about 30, 40, 45, 50 or 55 and about 60, 65, 70, 75, 80, 90 or 100 grams/m².

A suitable substrate may be a carded nonwoven comprising a 40/60 blend of viscose fibers and polypropylene fibers having a basis weight of 58 grams/m² as available from Suominen of Tampere, Finland as FIBRELLA 3160. Another suitable material for use as a substrate may be SAWATEX 2642 as available from Sandler AG of Schwarzenbach/Salle, Germany. Yet another suitable material for use as a substrate may have a basis weight of from about 50 grams/m² to about 60 grams/m² and have a 20/80 blend of viscose fibers and polypropylene fibers. The substrate may also be a 60/40 blend of pulp and viscose fibers. The substrate may also be formed from any of the following fibrous webs such as those available from the J.W. Suominen Company of Finland, and sold under the FIBRELLA trade name. For example, FIBRELLA 3100 is a 62 gsm nonwoven web comprising 50% 1.5 denier polypropylene fibers and 50% 1.5 denier viscose fibers. In both of these commercially available fibrous webs, the average fiber length is about 38 mm. Additional fibrous webs available from Suominen may include a 62 gsm nonwoven web comprising 60% polypropylene fibers and 40% viscose fibers; a fibrous web comprising a basis weight from about 50 or 55 to about 58 or 62 and comprising 60% polypropylene fibers and 40% viscose fibers; and a fibrous web comprising a basis weight from about 62 to about 70 or 75 gsm. The latter fibrous web may comprise 60% polypropylene fibers and 40% viscose fibers. The substrate may also be a 60 gsm nonwoven comprising a 40/60 blend of pulp and lyocell fibers.

In one embodiment of the present invention the surface of substrate may be essentially flat. In another embodiment of the present invention the surface of the substrate may optionally contain raised and/or lowered portions. These can be in the form of logos, indicia, trademarks, geometric patterns, images of the surfaces that the substrate is intended to clean (i.e., infant's body, face, etc.). They may be randomly arranged on the surface of the substrate or be in a repetitive pattern of some form.

In another embodiment of the present invention the substrate may be biodegradable. For example, the substrate could be made from a biodegradable material such as a polyesteramide, or a high wet strength cellulose.

Composition

The substrate may further comprise a soothing and/or cleansing composition. The composition impregnating the substrate is commonly and interchangeably called lotion, soothing lotion, soothing composition, oil-in-water emulsion composition, emulsion composition, emulsion, cleaning or cleansing lotion or composition. The composition may be suitable for a purpose selected from the group consisting of cleansing, skin soothing, moisturizing, exfoliating, and combinations thereof. All those terms are hereby used interchangeably. The composition may generally comprise the following optional ingredients: emollients, surfactants and/or an emulsifiers, soothing agents, rheology modifiers, preservatives, or more specifically a combination of preservative compounds acting together as a preservative system and water.

It is to be noted that some compounds can have a multiple function and that all compounds are not necessarily present in the composition of the invention. The composition may be a oil-in-water emulsion. The pH of the composition may be from about pH 3, 4 or 5 to about pH 7, 7.5, or 9.

Examples of compositions that may be used may be found in the Examples section as Examples A through E.

Emollient

In the substrates of the present invention, emollients may (1) improve the glide of the substrate on the skin, by enhancing the lubrication and thus decreasing the abrasion of the skin, (2) hydrate the residues (for example, fecal residues or dried urine residues), thus enhancing their removal from the skin, (3) hydrate the skin, thus reducing its dryness and irritation while improving its flexibility under the wiping movement, and (4) protect the skin from later irritation (for example, caused by the friction of underwear) as the emollient is deposited onto the skin and remains at its surface as a thin protective layer.

In one embodiment, emollients may be silicone based. Silicone based emollients may be organo-silicone based polymers with repeating siloxane (Si—O) units. Silicone-based emollients of the present invention may be hydrophobic and may exist in a wide range of possible molecular weights. They may include linear, cyclic and cross-linked varieties. Silicone oils may be chemically inert and may have a high flash point. Due to their low surface tension, silicone oils may be easily spreadable and may have high surface activity. Examples of silicon oil may include: cyclomethicones, dimethicones, phenyl-modified silicones, alkyl-modified silicones, silicones resins and combinations thereof.

Other useful emollients can be unsaturated esters or fatty esters. Examples of unsaturated esters or fatty esters of the present invention include: caprylic capric triglycerides in combination with Bis-PEG/PPG-16/16 PEG/PPG-16/16 dimethicone and C₁₂-C₁₅ alkylbenzoate and combinations thereof.

A relatively low surface tension may act more efficiently in the composition. Surface tension lower than about 35 mN/m, or even lower than about 25 mN/m. In certain embodiments, the emollient may have a medium to low polarity. Also, the emollient of the present invention may have a solubility parameter between about 5 and about 12, or even between about 5 and about 9. The basic reference of the evaluation of surface tension, polarity, viscosity and spreadability of emollient can be found under Dietz, T., Basic properties of cosmetic oils and their relevance to emulsion preparations. SÖFW-Journal, July 1999, pages 1-7.

Emulsifier

The composition may also include an emulsifier such as those forming oil-in-water emulsions. The emulsifier can be a mixture of chemical compounds and include surfactants. The emulsifier may be a polymeric emulsifier or a non polymeric one.

The emulsifier may be employed in an amount effective to emulsify the emollient and/or any other non-water-soluble oils that may be present in the composition, such as an amount ranging from about 0.5%, 1%, or 4% to about 0.001%, 0.01%, or 0.02% (based on the weight emulsifiers over the weight of the composition). Mixtures of emulsifiers may be used.

Emulsifiers for use in the present invention may be selected from the group consisting of alkylpolylglucosides, decylpolyglucoside, fatty alcohol or alkoxylated fatty alcohol phosphate esters (e.g., trilaureth-4 phosphate), sodium trideceth-3 carboxylate, or a mixture of caprylic capric triglyceride and Bis-PEG/PPG-16/16 PEG/PPG-16/16 dimethicone, polysorbate 20, and combinations thereof.

Rheology Modifier

Rheology modifiers are compounds that increase the viscosity of the composition at lower temperatures as well as at process temperatures. Each of these materials may also provide “structure” to the compositions to prevent settling out (separation) of insoluble and partially soluble components. Other components or additives of the compositions may affect the temperature viscosity/rheology of the compositions.

In addition to stabilizing the suspension of insoluble and partially soluble components, the rheology modifiers of the invention may also help to stabilize the composition on the substrate and enhance the transfer of lotion to the skin. The wiping movement may increase the shear and pressure therefore decreasing the viscosity of the lotion and enabling a better transfer to the skin as well as a better lubrication effect.

Additionally, the rheology modifier may help to preserve a homogeneous distribution of the composition within a stack of substrates. Any composition that is in fluid form has a tendency to migrate to the lower part of the wipes stack during prolonged storage. This effect creates an upper zone of the stack having less composition than the bottom part. This is seen as a sign of relatively low quality by the users.

Preferred rheology modifiers may exhibit low initial viscosity and high yield. Particularly suited are rheology modifiers such as, but not limited to:
Blends of material as are available from Uniqema GmbH&Co. KG, of Emmerich, Germany under the trade name ARLATONE. For instance, ARLATONE V-175 which is a blend of sucrose palmitate, glyceryl stearate, glyceryl stearate citrate, sucrose, mannan, and xanthan gum and Arlatone V-100 which is a blend of steareth-100, steareth-2, glyceryl stearate citrate, sucrose, mannan and xanthan gum.
Blends of materials as are available from Seppic France of Paris, France as SIMULGEL. For example, SIMULGEL NS which comprises a blend of hydroxyethylacrylate/sodium acryloyldimethyl taurate copolymer and squalane and polysorbate 60, sodium acrylate/sodium acryloyldimethyltaurate copolymer and polyisobutene and caprylyl capryl glucoside, acrylate copolymers, such as but not limited to acrylates/acrylamide copolymers, mineral oil, and polysorbate 85.
Acrylate homopolymers, acrylate crosspolymers, such as but not limited to, acrylate/C10-30 alkyl acrylate crosspolymers, carbomers, such as but not limited to acrylic acid cross linked with one or more allyl ether, such as but not limited to allyl ethers of pentaerythritol, allyl ethers of sucrose, allyl ethers of propylene, and combinations thereof as are available are available as the Carbopol® 900 series from Noveon, Inc. of Cleveland, Ohio (e.g., Carbopol® 954).
Naturally occurring polymers such as xanthan gum, galactoarabinan and other polysaccharides.
Combinations of the above rheology modifiers.

Examples, of commercially available rheology modifiers include but are not limited to, Ultrez-10, a carbomer, and Pemulen TR-2, an acrylate crosspolymers, both of which are available from Noveon, Cleveland Ohio, and Keltrol, a xanthan gum, available from CP Kelco San Diego Calif.

Rheology modifiers imparting a low viscosity may be used. Low viscosity is understood to mean viscosity of less than about 10,000 cps at about 25 degrees Celsius of a 1% aqueous solution. The viscosity may be less than about 5,000 cps under the same conditions. Further, the viscosity may be less than about 2000 cps or even less than about 1,000 cps. Other characteristics of emulsifiers may include high polarity and a non-ionic nature.

Rheology modifiers, when present may be used in the present invention at a weight/weight % (w/w) from about 0.01%, 0.015%, or 0.02% to about 1%, 2%, or 3%.

Preservative

The need to control microbiological growth in personal care products is known to be particularly acute in water based products such as oil-in-water emulsions and in pre-impregnated substrates such as baby wipes. The composition may comprise a preservative or more preferably a combination of preservatives acting together as a preservative system. Preservatives and preservative systems are used interchangeably in the present document to indicate one unique or a combination of preservative compounds. A preservative is understood to be a chemical or natural compound or a combination of compounds reducing the growth of microorganisms, thus enabling a longer shelf life for the pack of wipes (opened or not opened) as well as creating an environment with reduced growth of microorganisms when transferred to the skin during the wiping process.

Preservatives of the present invention can be defined by 2 key characteristics: (i) activity against a large spectrum of microorganisms, that may include bacteria and/or molds and/or yeast, preferably all three categories of microorganisms together and (2) killing efficacy and/or the efficacy to reduce the growth rate at a concentration as low as possible.

The spectrum of activity of the preservative of the present invention may include bacteria, molds and yeast. Ideally, each of such microorganisms is killed by the preservative. Another mode of action to be contemplated is the reduction of the growth rate of the microorganisms without active killing. Both actions however result in a drastic reduction of the population of microorganisms.

Suitable materials include, but are not limited to a methylol compound, or its equivalent, an iodopropynyl compound and mixtures thereof. Methylol compounds release a low level of formaldehyde when in water solution that has effective preservative activity. Exemplary methylol compounds include but are not limited to: diazolidinyl urea (GERMALL® II as is available from International Specialty Products of Wayne, N.J.) N-[1,3-bis(hydroxy-methyl)-2,5-dioxo-4-imidazolidinyl]-N,N′-bis(hydroxymethyl) urea, imidurea (GERMALL® 115 as is available from International Specialty Products of Wayne, N.J.), 1,1-methylene bis[3-[3-(hydroxymethyl)-2,5-dioxo-4-imidazolidinyl]urea]; 1,3-dimethylol-5,5-dimethyl hydantoin (DMDMH), sodium hydroxymethyl glycinate (SUTTOCIDE® A as is available from International Specialty Products of Wayne, N.J.), and glycine anhydride dimethylol (GADM). Methylol compounds can be effectively used at concentrations (100% active basis) between about 0.025% and about 0.50%. A preferred concentration (100% basis) is about 0.075%. The iodopropynyl compound provides antifungal activity. An exemplary material is iodopropynyl butyl carbamate as is available from Clariant UK, Ltd. of Leeds, The United Kingdom as NIPACIDE IPBC. A particularly preferred material is 3-iodo-2-propynylbutylcarbamate. Iodopropynyl compounds can be used effectively at a concentration between about 0% and about 0.05%. A preferred concentration is about 0.009%. A particularly preferred preservative system of this type comprise a blend of a methylol compound at a concentration of about 0.075% and a iodopropynyl compound at a concentration of about 0.009%.

In another embodiment, the preservative system may comprise simple aromatic alcohols (e.g., benzyl alcohol). Materials of this type have effective anti-bacterial activity. Benzyl alcohol is available from Symrise, Inc. of Teterboro, N.J.

In another embodiment, the preservative may be a paraben antimicrobial selected from the group consisting of methylparaben, ethylparaben, propylparaben, butylparaben, isobutylparaben or combinations thereof.

Chelators (e.g., ethylenediamine tetraacetic acid and its salts) may also be used in preservative systems as a potentiator for other preservative ingredients.

The preservative composition can moreover provide a broad anti-microbial effect without the use of formaldehyde donor derived products. These traditional formaldehyde based preservative products have been widely used in the past but are now no longer permitted in a number of countries for products intended for human use.

Optional Components of the Composition

The composition may optionally include adjunct ingredients. Possible adjunct ingredients may be selected from a wide range of additional ingredients such as, but not limited to soothing agents, perfumes and fragrances, texturizers, colorants, medically active ingredients, in particular healing actives and skin protectants.

Optional soothing agents may be (a) ethoxylated surface active compounds, more preferably those having an ethoxylation number below about 60, (b) polymers, more preferably polyvinylpyrrolidone (PVP) and/or N-vinylcaprolactam homopolymer (PVC), and (c) phospholipids, more preferably phospholipids complexed with other functional ingredients as e.g., fatty acids, organosilicones.

The soothing agents may be selected from the group comprising PEG-40 hydrogenated castor oil, sorbitan isostearate, isoceteth-20, sorbeth-30, sorbitan monooleate, coceth-7, PPG-1-PEG-9 lauryl glycol ether, PEG-45 palm kernel glycerides, PEG-20 almond glycerides, PEG-7 hydrogenated castor oil, PEG-50 hydrogenated castor oil, PEG-30 castor oil, PEG-24 hydrogenated lanolin, PEG-20 hydrogenated lanolin, PEG-6 caprylic/capric glycerides, PPG-1 PEG-9 lauryl glycol ether, lauryl glucoside polyglyceryl-2 dipolyhydroxystearate, sodium glutamate, polyvinylpyrrolidone, N-vinylcaprolactam homopolymer, sodium coco PG-dimonium chloride phosphate, linoleamidopropyl PG-dimonium chloride phosphate, dodium borageamidopropyl PG-dimonium chloride phosphate, N-linoleamidopropyl PG-dimonium chloride phosphate dimethicone, cocamidopropyl PG-dimonium chloride phosphate, stearamidopropyl PG-dimonium chloride phosphate and stearamidopropyl PG-dimonium chloride phosphate (and) cetyl alcohol, and combinations thereof. A particularly preferred soothing agent is PEG-40 hydrogenated castor oil as is available from BASF of Ludwigshafen, Germany as Cremophor CO 40.

Method of Making Molded Fibrous Structure

Generally, the process of the present invention for making a fibrous structure may be described in terms of initially forming a fibrous web having a plurality of synthetic fibers and/or natural fibers. Layered deposition of the fibers, synthetic and natural, is also contemplated by the present invention. The fibrous web can be formed in any conventional fashion and may be any nonwoven web that may be suitable for use in a hydromolding process. The fibrous web may consist of any web, mat, or batt of loose fibers disposed in any relationship with one another or in any degree of alignment, such as might be produced by carding, air laying, spunmelting (including meltblowing and spunlaying), coforming and the like.

In the present invention, conducting the carding, spunmelting, spunlaying, meltblowing, coforming, air laying or other formation processes concurrently with the fibers contacting a forming member may produce a fibrous web. The process of the present invention may involve subjecting the fibrous web to a hydroentanglement process while the fibrous web is in contact with the forming member. The hydroentanglement process (also known as spunlacing or spunbonding) is a known process of producing nonwoven webs, and involves laying down a matrix of fibers, for example as a carded web or an air laid web, and entangling the fibers to form a coherent web. Entangling is typically accomplished by impinging the matrix of fibers with high pressure liquid (typically water) from at least one, at least two, or a plurality of suitably placed water jets. The pressure of the liquid jets, as well as the orifice size and the energy imparted to the fibrous structure preform by the water jets, may be the same as those of a conventional hydroentangling process. Typically, entanglement energy may be about 0.1 kwh/kg. While other fluids can be used as the impinging medium, such as compressed air, water is the preferred medium. The fibers of the web are thus entangled, but not physically bonded one to another. The fibers of a hydroentangled web, therefore, have more freedom of movement than fibers of webs formed by thermal or chemical bonding. Particularly when lubricated by wetting as a pre-moistened wet wipe, such spunlaced webs provide webs having very low bending torques and low moduli, thereby achieving softness and suppleness.

Additional information on hydroentanglement can be found in U.S. Pat. Nos. 3,485,706 issued on Dec. 23, 1969, to Evans; 3,800,364 issued on Apr. 2, 1974, to Kalwaites; 3,917,785 issued on Nov. 4, 1975, to Kalwaites; 4,379,799 issued on Apr. 12, 1983, to Holmes; 4,665,597 issued on May 19, 1987, to Suzuki; 4,718,152 issued on Jan. 12, 1988, to Suzuki; 4,868,958 issued on Sep. 26, 1989, to Suzuki; 5,115,544 issued on May 26, 1992, to Widen; and 6,361,784 issued on Mar. 26, 2002, to Brennan.

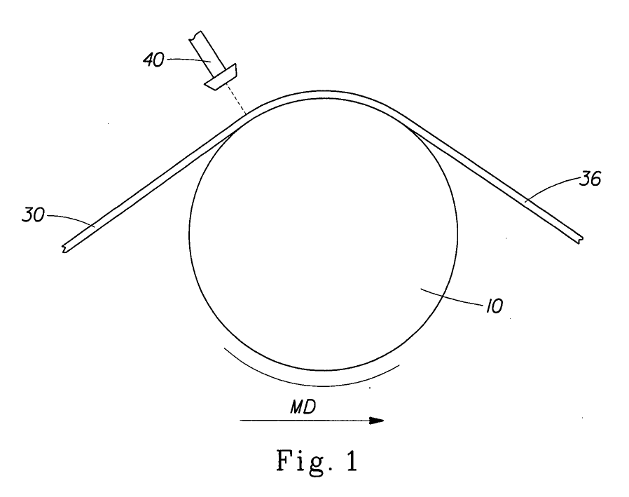

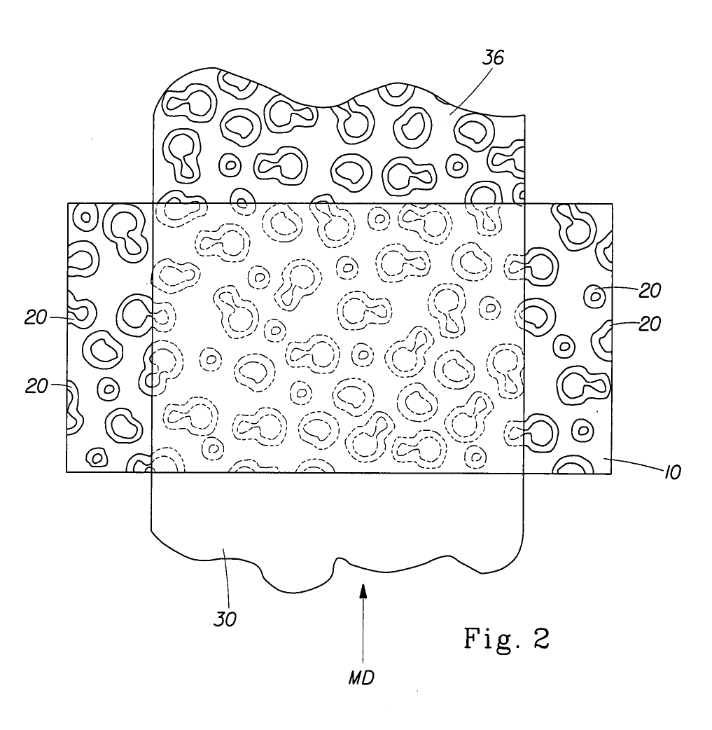

After the fibrous web has been formed, it can be subjected to additional process steps, such as, hydromolding (also known as molding, hydroembossing, hydraulic needlepunching, etc.). FIG. 1 illustrates a side view of a molding member 10 with a fibrous web 30 being conveyed over the top of the molding member 10. A single jet 40, or multiple jets, may be utilized. Water or any other appropriate fluid medium may be ejected from the jet 40 to impact the fibrous web 30. The fluid may impact the fibrous web in a continuous flow or noncontinuous flow. The molding member 10 may comprise a molding pattern (as exemplified in FIG. 2). The molding pattern may comprise raised areas, lowered areas, or combinations thereof. As the fluid from the jet(s) 40 impacts the fibrous web 30, the fibrous web 30 may conform to the molding pattern. The fluid may “push” portions of the fibrous web 30 into lowered areas of the pattern. The result may be a molded fibrous structure 36.

FIG. 2 illustrates a top view of a molding member 10 with a fibrous web 30 conveyed over the top of the molding member 10. A pattern 20 may be molded onto the fibrous web 30 by a hydromolding process. In such a process, fluid may be directed towards the fibrous web 30 in such as manner as to impact the fibrous web 30 causing it to conform to the pattern 20 on the molding member 10 resulting in a molded fibrous structure 36.

Following the hydromolding of the pattern onto the fibrous web, the resulting molded fibrous structure may continue to be processed in any method known to one of ordinary skill to covert the molded fibrous structure to a substrate suitable for use as a wipe. This may include, but is not limited to, slitting, cutting, perforating, folding, stacking, interleaving, lotioning and combinations thereof.

By molding the fibrous web, it can gain additional aesthetics making the fibrous web particularly suitable and pleasing for use as a wipe. Hydromolding of fibrous structures and of substrates useful as wipes is known in the art. Hydromolding, as may be applied to substrates useful as wipes, may include a number of decorative patterns with high levels of molding (i.e. about 50% or more of the substrate includes hydromolded regions). Such patterns may include regular arrays of small geometric shapes (i.e. circles), regular repeating patterns of lines, and curves, images of animals, etc. Such patterns may include high levels of hydromolding over the face of the substrate in order to impart the perception of texture impression.

Other beneficial physical characteristics may be imparted to the fibrous web by molding. Specifically, molding a fibrous web may have an effect on the fluid uptake and retention capabilities of the molded fibrous structure. Without being bound by theory, it is believed that fluid uptake may be a function of both the total fluid holding capacity (defined by capillary void space) of the fibrous structure and the ease with which the impinging liquid can enter the capillary void spaces.

Without being bound by theory, it is believed that an unmolded fibrous structure may comprise of a plurality of capillary void spaces. The total effective capillary void space volume of the fibrous structure may determine the total fluid holding capacity of the fibrous structure. However, introducing fluid from free space into the capillary void spaces of the fibrous structure requires an abrupt transition of the fluid from free space to the bound space of the capillary void spaces of the fibrous structure.

Hydromolding the fibrous webs may result in a disruption of the capillary void spaces, yielding a more “open” void space structure. The open void spaces created by the hydromolding, however, may not contribute to the total fluid holding capacity of the fibrous structure to the same extent as does the capillary void space of the unmolded regions.

However, the open void space volume created by the hydromolded regions may contribute positively to the ease with which the fibrous structure is able to acquire an impinging liquid. Specifically, the larger voids and more open “conduits” within the fibrous structure void space structure may allow for an increased flow of fluid into and through the open void spaces created by the hydromolded regions. The increased flow of fluid into and through the hydromolded regions may help “channel” the liquid into the capillary void spaces of the unmolded regions by obviating the abrupt transition of the liquid from free space to the bound space of the capillary void space of the fibrous structure.

The optimal fluid uptake and acquisition by the fibrous structure may be achieved through a balancing of the hydromolded regions, which may facilitate the uptake, and the unmolded regions, which may retain the fluid. In the extreme of a fully hydromolded structure (i.e. 100% molded regions), the flow of the liquid into and through the substrate would be most highly facilitated, however, there would be no capacity for the fibrous structure to retain the fluid. Alternately, in the extreme of an unmolded structure (i.e. 100% unmolded regions), the fluid holding capacity of the fibrous structure would be maximized, but the ability of the fibrous structure to acquire the fluid would be compromised. Only in the right balance of molded regions and unmolded regions may the fluid handling of the fibrous structure optimized.

As such, an optimization in the amount of molding of the fibrous structure may be beneficial in aiding the molded fibrous structure to maintain and/or improve its fluid uptake and retention. It has also been discovered that no molding of a fibrous structure may result in reduced fluid uptake and retention relative to the optimum. It has also been discovered that greater than about 50% molded area of a fibrous structure may result in reduced fluid uptake and retention relative to the optimum. About or less than about 45% molded area may be present on the molded fibrous structure. More than about 0% molded area may be present on the molded fibrous structure. The molded fibrous structure may comprise from about 5, 10, 13, 15, 17, 18, or 20% to about 25, 30, 35, 40, or 45% molded area. The amount of molded area may be measured by comparing the total area of the molding pattern present on the molding member versus the total area of the “flat” spaces (i.e., nonmolding pattern space) present on the molding member.

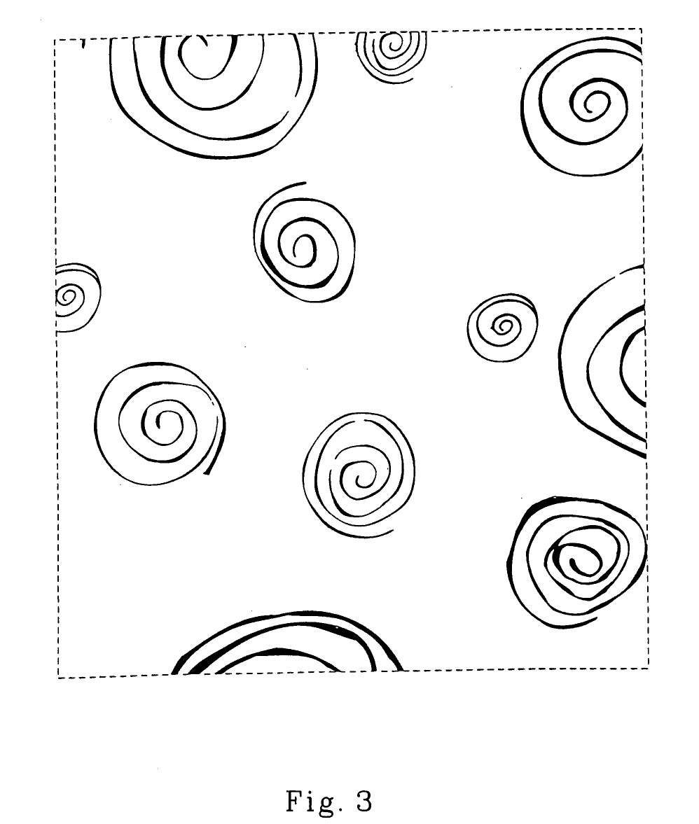

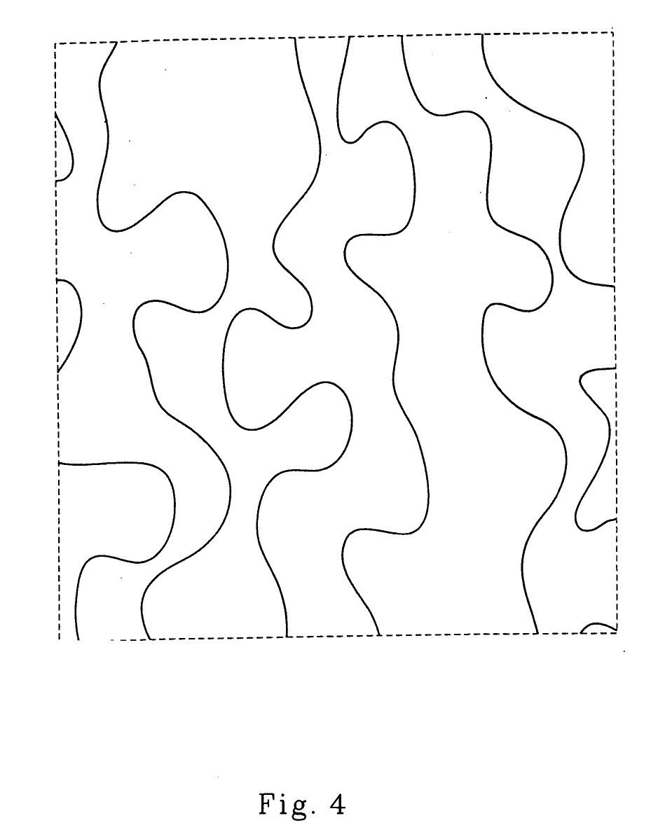

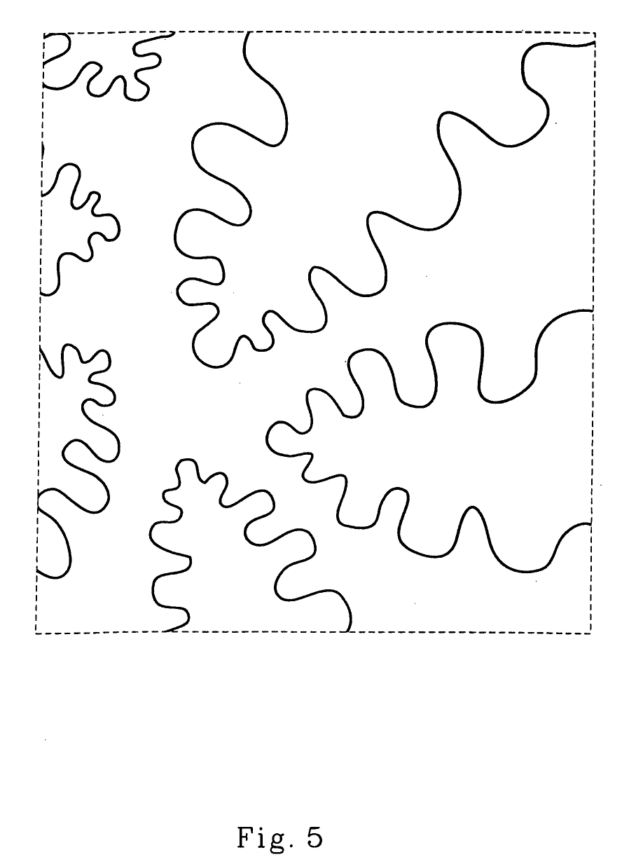

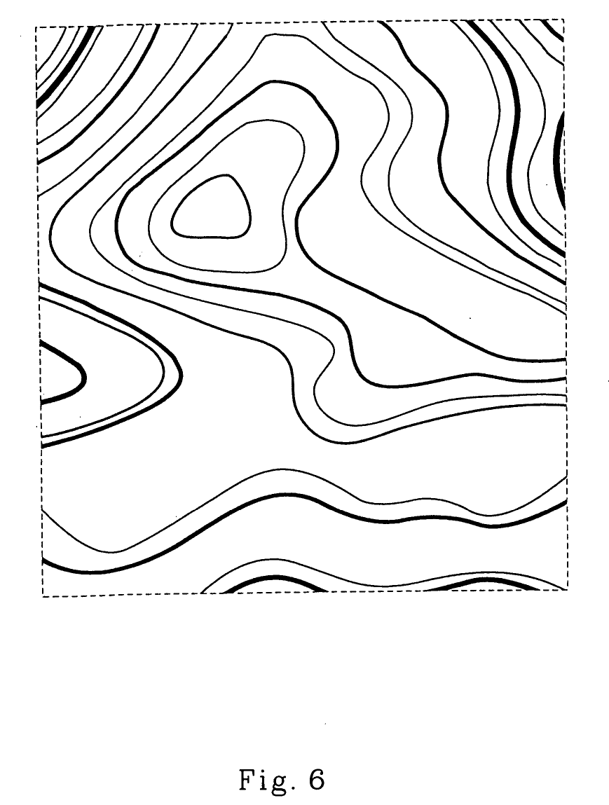

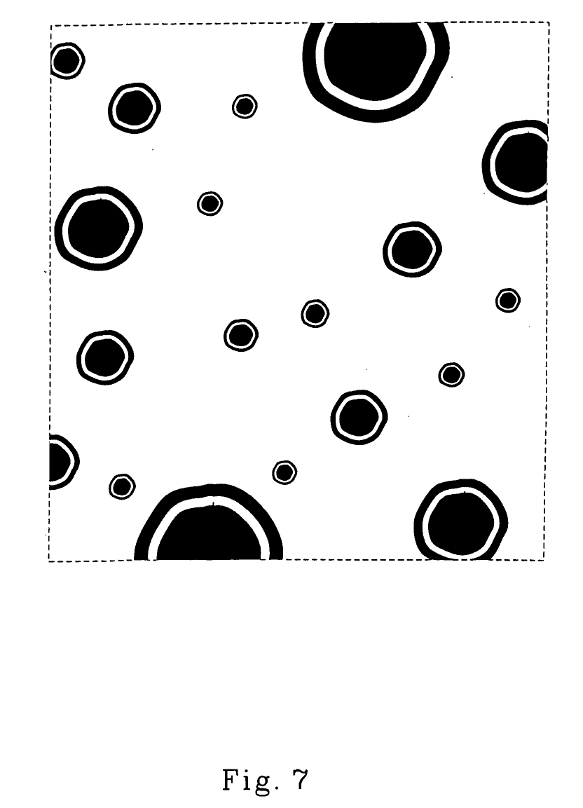

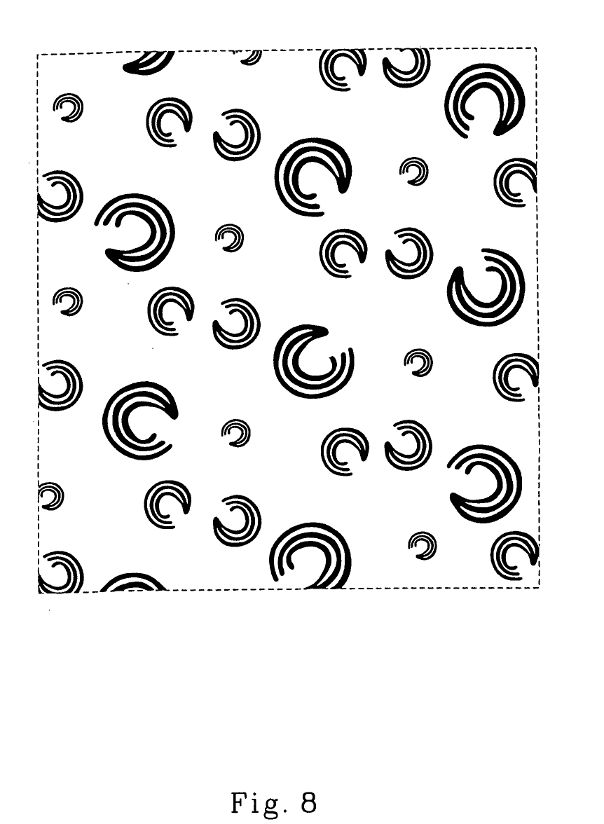

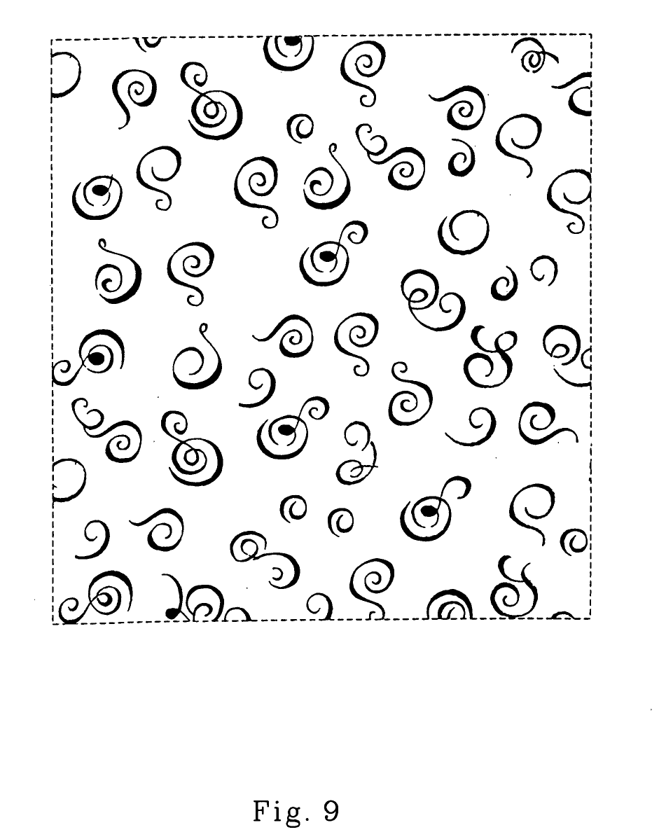

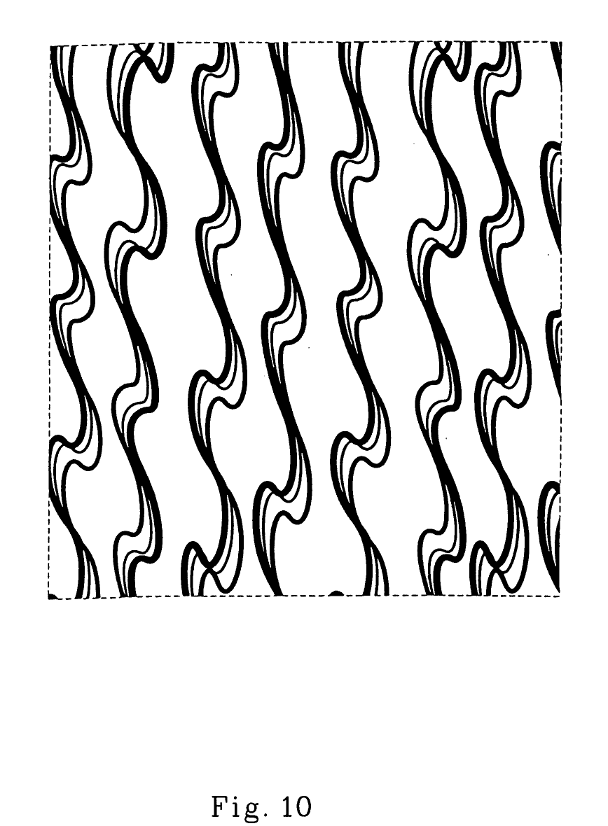

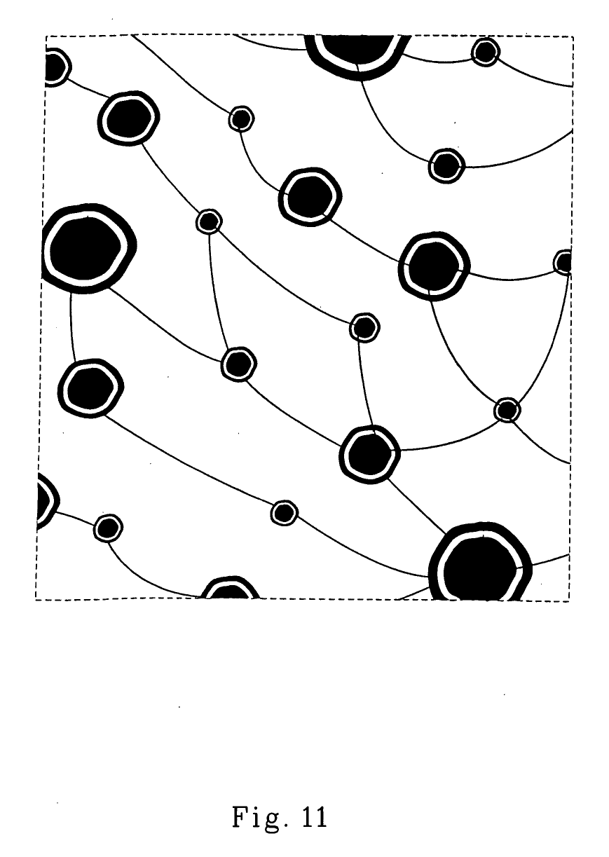

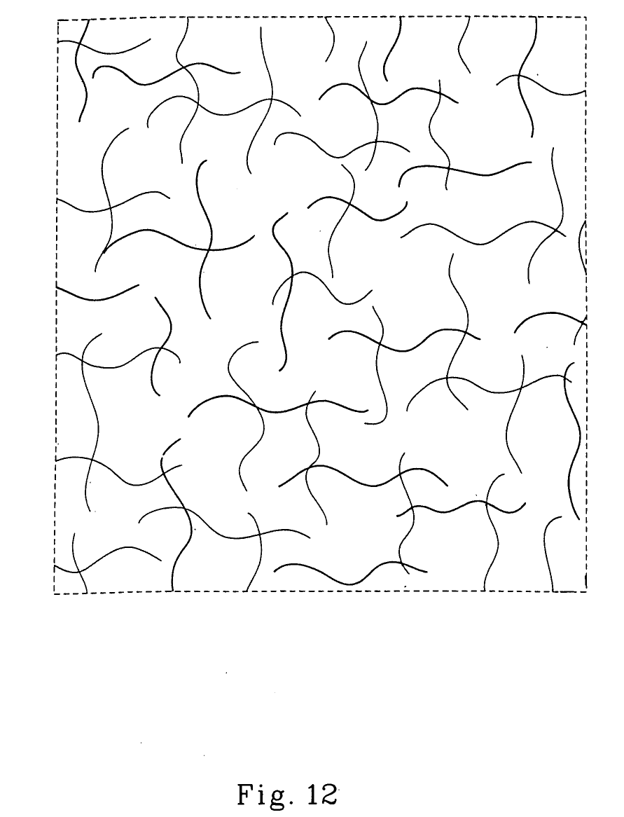

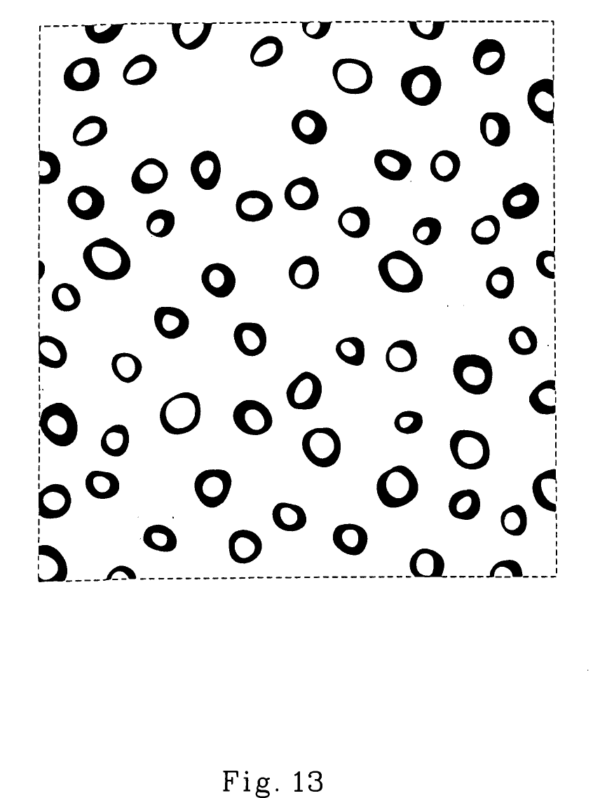

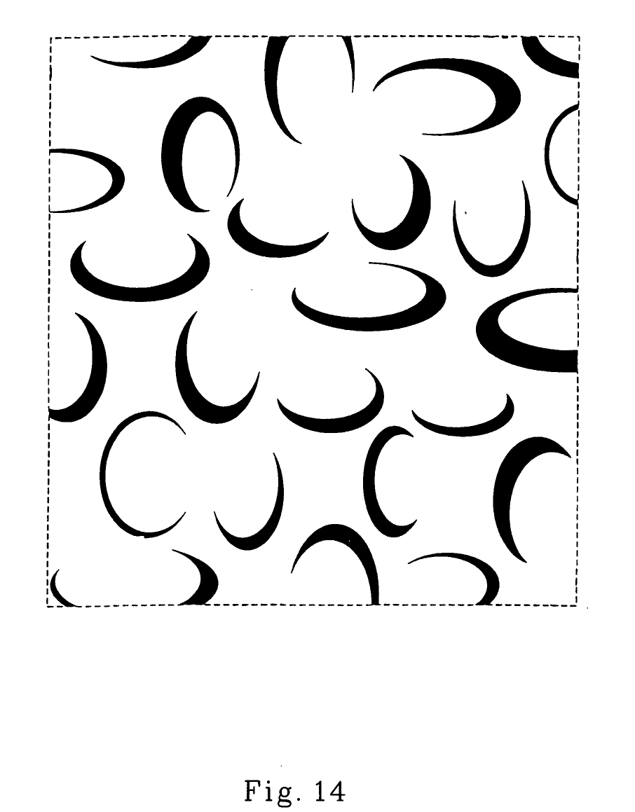

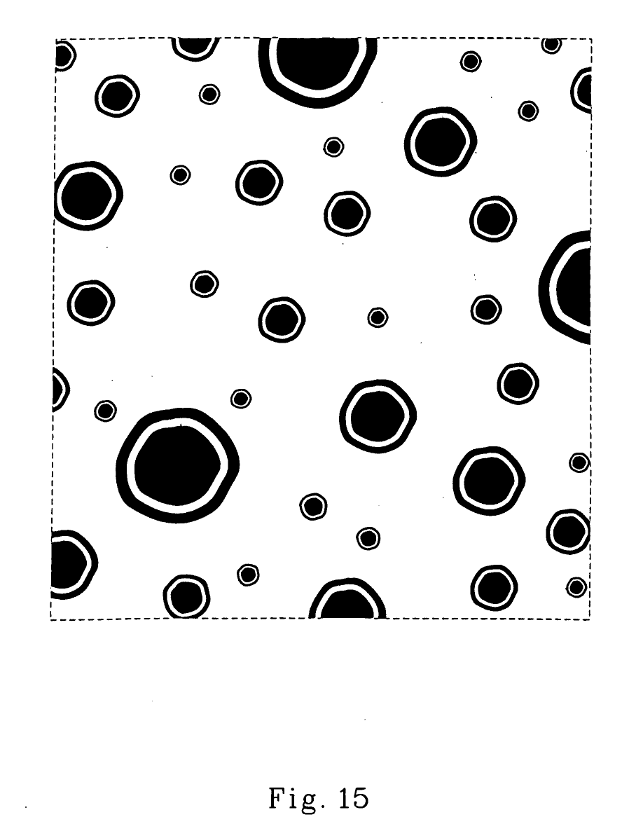

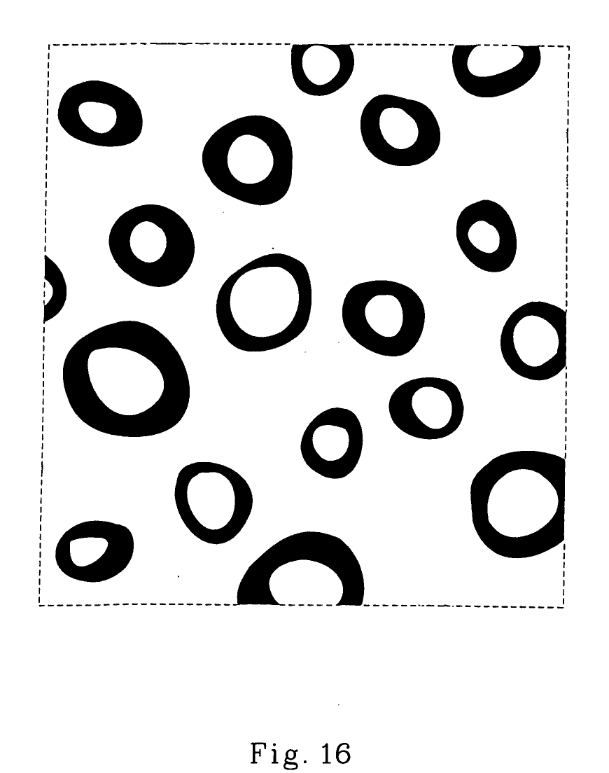

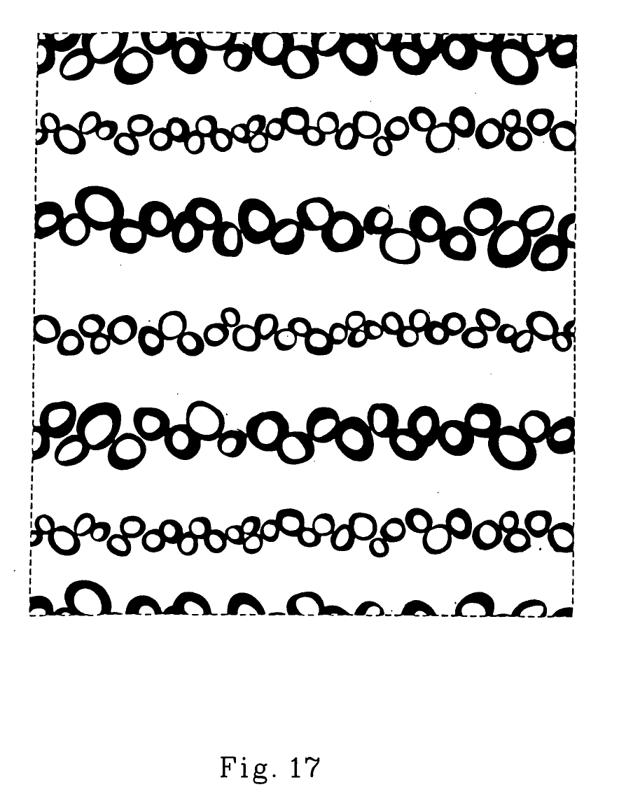

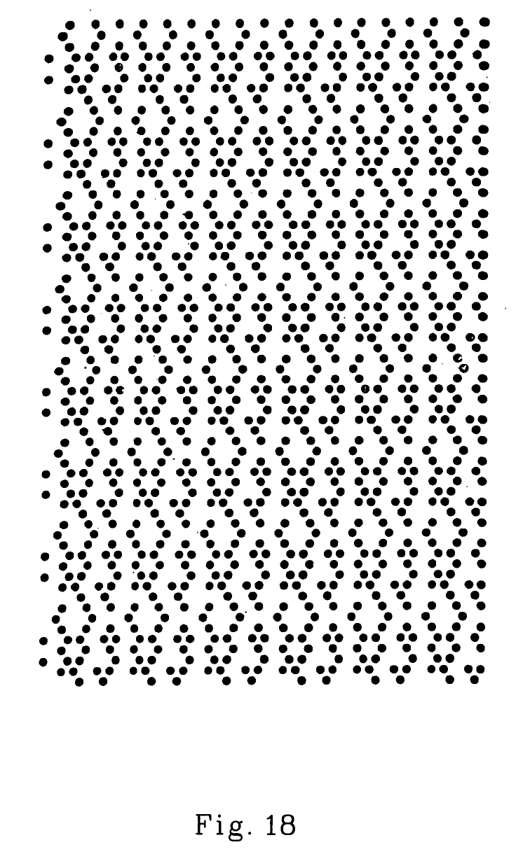

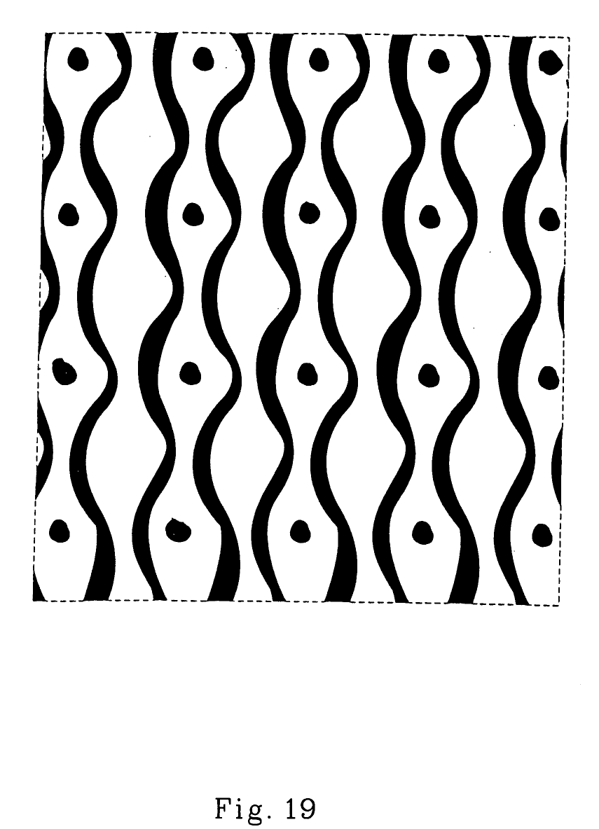

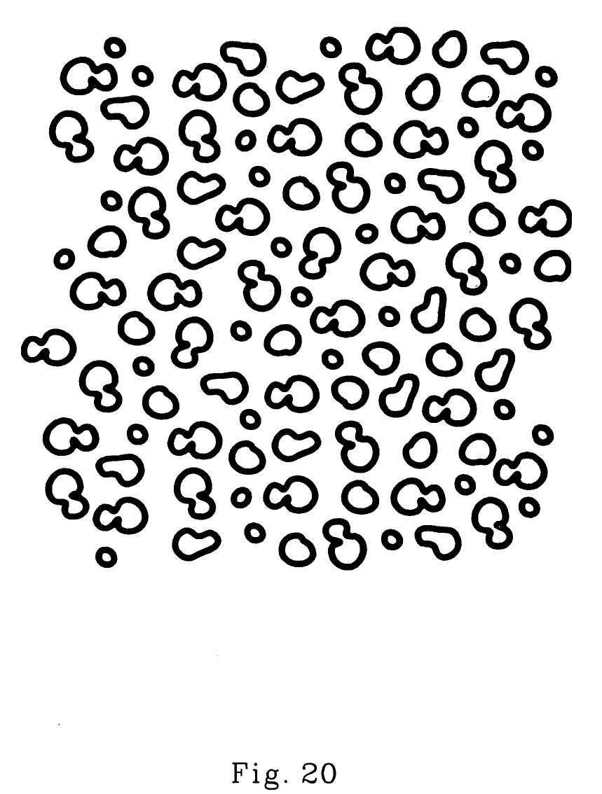

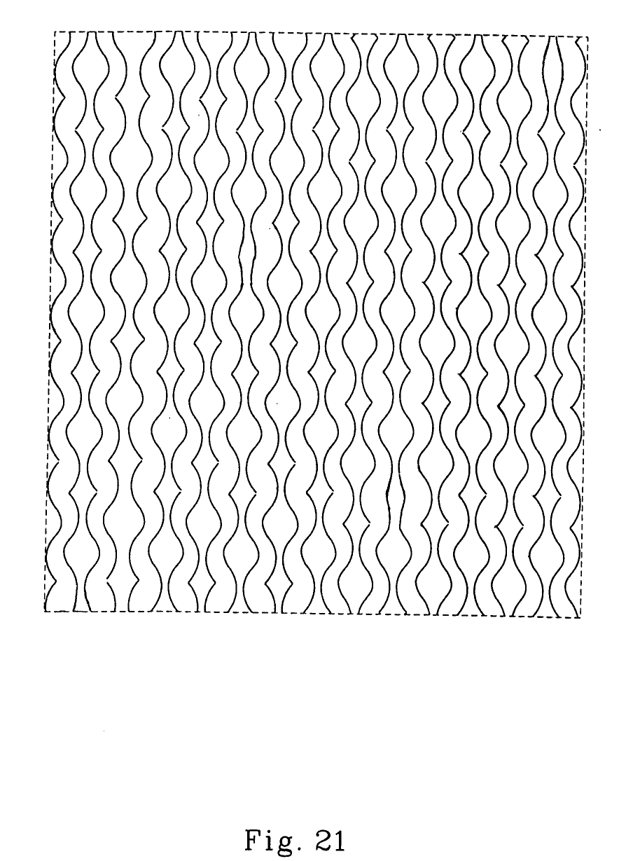

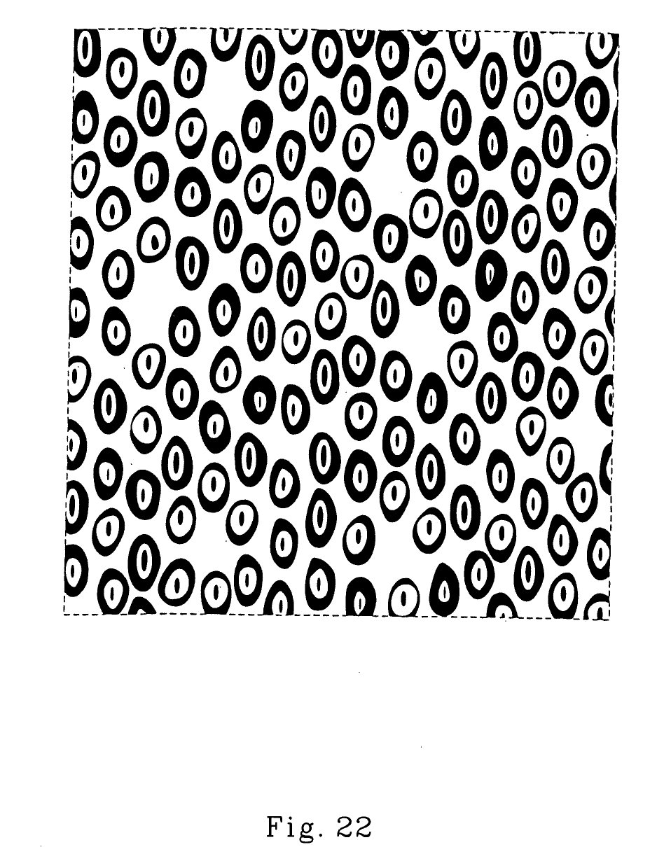

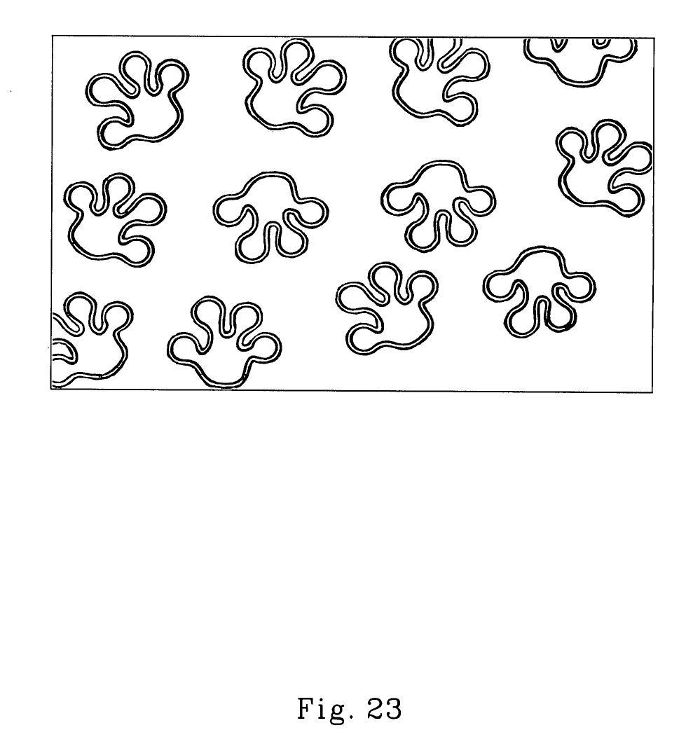

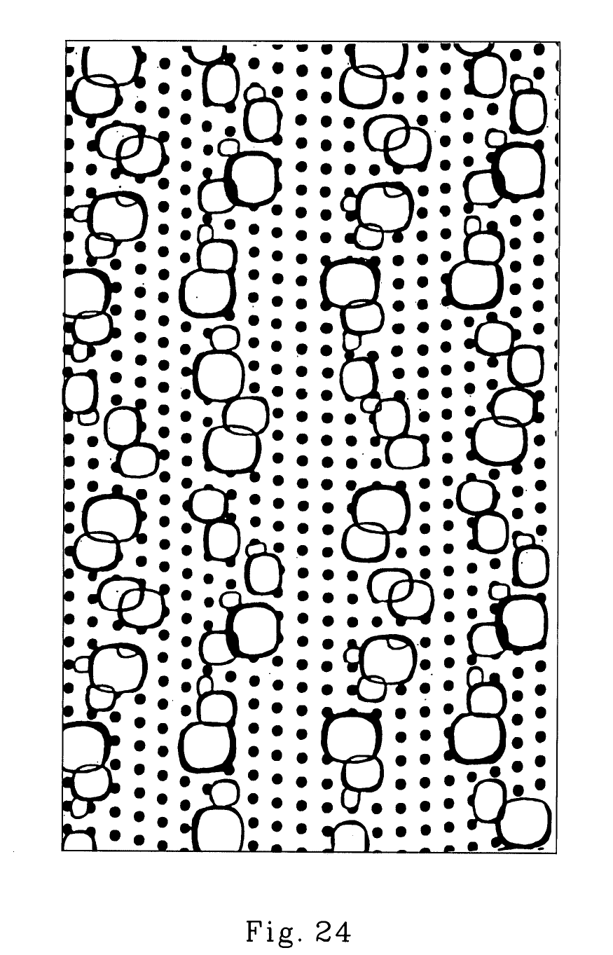

FIGS. 3 through 24 illustrate various molding patterns comprising various amounts of molded areas. FIG. 3 illustrates a swirl molding pattern comprising about 5% molded area. FIG. 4 illustrates a puzzle molding pattern comprising about 5% molded area. FIG. 5 illustrates a molding pattern comprising outlines of leaves comprising about 5% molded area. FIG. 6 illustrates a curving line molding pattern comprising from about 5 to about 10% molded area. FIG. 7 illustrates a circle molding pattern comprising about 10% molded area. FIG. 8 illustrates a multi-line circle molding pattern comprising about 10% molded area. FIG. 9 illustrates a curly cue molding pattern comprising about 10% molded area. FIG. 10 illustrates an overlapping wavy line molding pattern comprising about 10% molded area. FIG. 11 illustrates a connected circle molding pattern comprising about 12% molded area. FIG. 12 illustrates a cross hatch mark molding pattern comprising about 15% molded area. FIG. 13 illustrates an irregular circle molding pattern comprising about 17% molded area. FIG. 14 illustrates a pebble molding pattern comprising about 20% molded area. FIG. 15 illustrates a circle molding pattern comprising about 20% molded area. FIG. 16 illustrates an irregular circle molding pattern comprising about 23% molded area. FIG. 17 illustrates a linear circle molding pattern comprising about 24% molded area. FIG. 18 illustrates a molding pattern comprising solid discrete molded elements arranged in an irregular pattern comprising about 25% molded area. FIG. 19 illustrates a molding pattern comprising waves and dots comprising about 27% molded area. FIG. 20 comprises hollow irregular circles comprising about 29% molded area. FIG. 21 illustrates a bubble line molding pattern comprising about 32% molded area. FIG. 22 illustrates a honeycomb molding pattern comprising about 38% molded area. FIG. 23 illustrates an embodiment of a molding pattern comprising paw prints and comprising from about 10 or 13% to about 18 or 20% molded area. FIG. 24 illustrates an embodiment of a molding pattern comprising soft squares and comprising from about 15% to about 17 or 20% molded area.

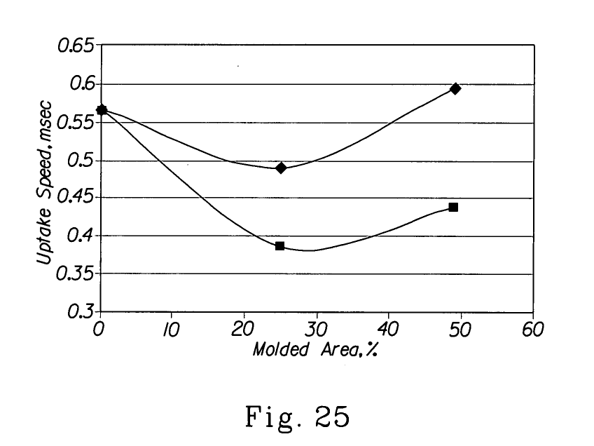

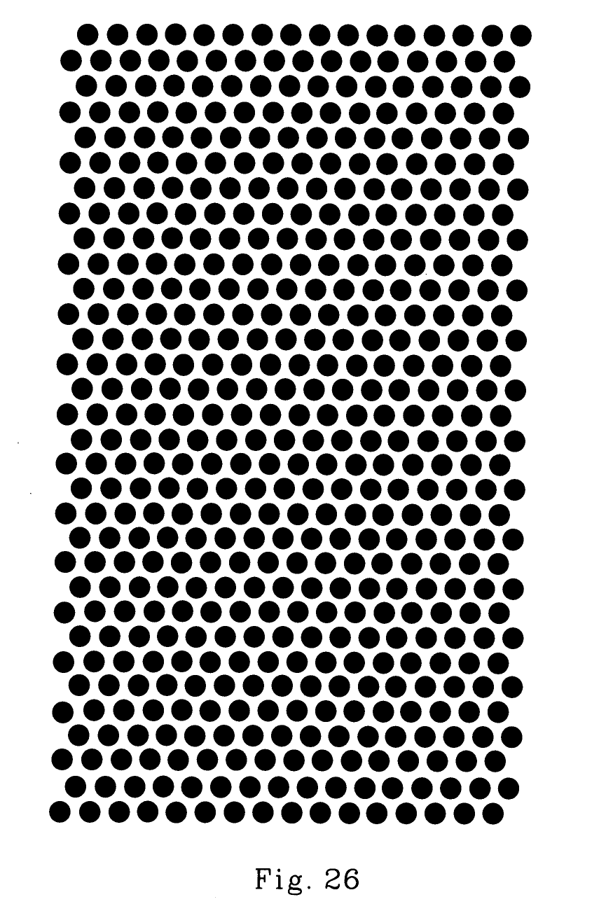

FIG. 25 illustrates the speed of fluid uptake, such as the composition of Example F, of two molded fibrous structures. FIG. 18 illustrates the molding pattern of both fibrous structures comprising about 25% molded area. FIG. 26 illustrates the molding pattern of both fibrous structures comprising about 49% molded area. The speed of fluid uptake increases as the percentage of molded area increases above about 0% and approaches 25% molded area. The speed of fluid uptake increases as the percentage of molded area decreases below about 50% and approaches 25%. The speed of fluid uptake may be greatest when the fibrous structure comprises from about 5, 10, 15 or 20% to about 25, 30, 35, 40 or 45% molded area. The first molded fibrous structure (represented by diamonds) comprises a 60/40 blend of polypropylene fibers and viscose fibers and has a basis weight of 58 gsm. With 0% molded area, the fibrous structure requires about 0.57 msec to uptake the fluid. With 49% molded area, the fibrous structure requires about 0.59 msec to uptake the fluid. With 25% molded area, the speed of fluid uptake is increased and the fibrous structure requires about 0.49 msec to uptake the fluid. It should be recognized by one of skill that speed of fluid uptake may be impacted by the fibrous composition of the fibrous structure. The second molded fibrous structure (represented by squares) comprises a 40/60 blend of pulp fibers and lyocell fibers and has a basis weight of 60 gsm. With 0% molded area, the fibrous structure requires about 0.57 msec to uptake the fluid. At 49% molded area, the fibrous structure requires about 0.44 msec to uptake the fluid. The speed of fluid uptake, however, is increased with 25% molded area wherein the fibrous structure requires about 0.39 msec to uptake the fluid. Therefore, while the speed of fluid uptake may be affected by the fibrous composition of the fibrous structures, it may be evident that the amount of molded area plays a role resulting in an increase in the speed of fluid uptake when the fibrous structure comprises greater than about 0% molded area and less than about 50% molded area. Fluid uptake may be determined according to the test method described herein.

However, to the extent that the fluid uptake of the molded fibrous structure is improved through the use of low levels of hydromolding, it is important to maintain the high texture impression of the fibrous structure, and resulting substrate, as if it is highly molded. The perceived texture impression of high level molding may provide a visual signal to the user that the substrate is soft, strong, flexible, and provides an improved cleansing benefit.

Various molding patterns may provide a user with a texture impression of a substrate. In the absence of high level molding, the challenge is to maintain the high texture impression with low level molding of the fibrous structure and resulting substrates. Without being bound by theory, it is believed that texture impression of a high level molded structure may be achieved by manipulating the size and the relative proximity of the molded elements. In one embodiment, larger molded elements spaced farther apart can create a high texture impression. In another embodiment, smaller molded elements placed closer together can create a high texture impression. However, smaller molded elements placed farther apart may not create a high texture impression.

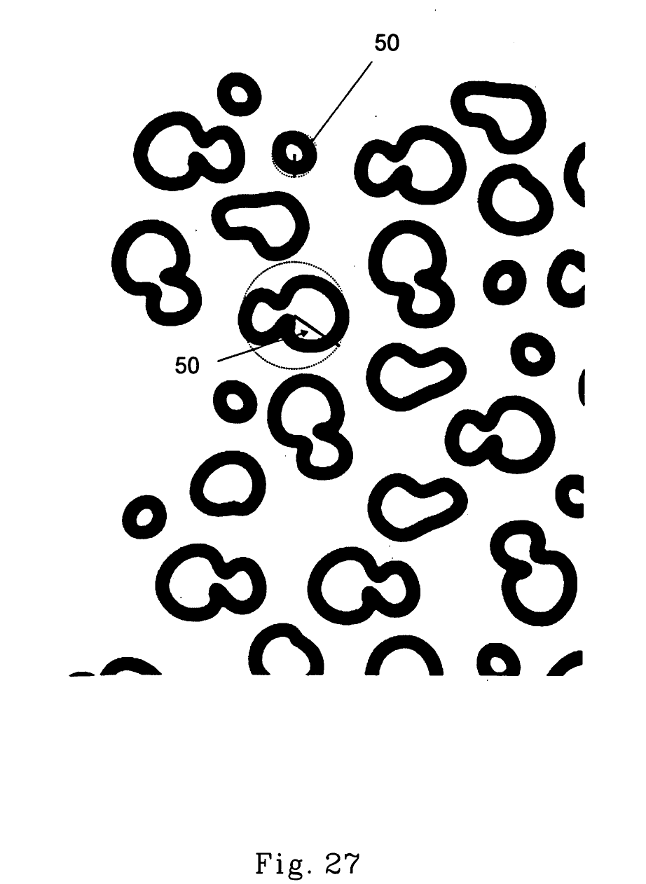

High texture impression may be a result of the size and relative proximity of the molded elements on the fibrous structure and resulting substrate. In one embodiment, a fibrous structure may comprise at least two molded elements. In such an embodiment, the smaller of the two elements may be circumscribed by the smallest possible circle that may be drawn around the molded element and completely encircle the molded element. The circumscribing circle may therefore comprise a radius that it may impart to the molded element. The radius provided by the circumscribing circle may be deemed a “radius unit.” “Radius Unit” refers herein to the distance that equals the radius of the smallest circumscribing circle that can be drawn around the smallest molded element that completely contains the molded element. FIG. 27 illustrates a radius unit 50 of a hollow irregular molded element. The circumscribed molded element may have as a nearest neighbor the second molded element. The circumscribed molded element may be within about 4 radius units of the second molded element. Two molded elements within about 4 radius units of each other may provide a high texture impression. In another embodiment, the circumscribed molded element may be within about 1, 1.5, 2, 2.5, 3, or 3.5 radius units of the second molded element. It should be realized that the circumscribing circles utilized to provide radius units to the molded elements may overlap.

It should be realized by one of skill in the art that fibrous structures comprising greater than about 50% molded area may already provide a user with a high texture impression. The high texture impression described herein may be for those fibrous structures comprising about or less than about 45% molded area. As noted above, a decrease in the amount of molded area may negatively impact a user's impression of the texture of a fibrous structure. The molding patterns described herein, and similar molding patterns, may provide a low level of molded area to a fibrous structure and simultaneously maintain a high texture impression.

A number of approaches to molding patterns can simultaneously deliver a low level of molding and high texture impression. In one embodiment, the molding pattern may comprise molded elements that are hollow (such as FIG. 20). As noted above, hollow may refer to a molded element that may be patterned to comprise an outline of a molded area enclosing an unmolded interior area. Multiple hollow molded elements may be present on the fibrous structure to provide the high texture impression. As the elements are hollow, though, the actual molded area of the fibrous structure may be low. Thus, the utilization of hollow molded elements may simultaneously provide for both high texture impression and an increase in the fluid uptake by the fibrous structure. It can be appreciated by one of skill in the art that if the molded elements are not hollow, and therefore are solid, that the pattern may include higher levels of hydromolding. The higher levels of hydromolding may not provide the fluid uptake advantages that may be associated with the use of lower levels of hydromolding. The fluid uptake benefits may be regained if the fibrous structure comprise a fewer number of solid molded elements. A fewer number of solid molded elements, however, may still not provide a high texture impression.

In another embodiment of molding patterns comprising hollow molded elements, the outline of the molded element area need not fully enclose the unmolded interior area. FIGS. 3 and 14 exemplify patterns comprising about 5% and about 20% molded area, respectively, in which the hollow molded elements do not fully enclose the interior unmolded area. Both molded patterns, however, may provide a high texture impression.

In another embodiment, the molding pattern may comprise molded elements that may be arranged in an irregular pattern to achieve a low level of hydromolding and simultaneously a high texture impression. FIG. 18 exemplifies a molding pattern that may use solid discrete molded elements in an irregular pattern to simultaneously achieve low level hydromolding (about 25% molded area) and texture impression of a highly molded fibrous structure.

In an alternate embodiment, high texture impression and the use of a low level of hydromolding may be achieved with a molding pattern comprising extended molded elements. FIG. 12 exemplifies a molding pattern comprising “cross-hatches” (about 15% molded area) in an overlapping and non-overlapping pattern.

### EXAMPLES

Examples A-C are examples of the fluid uptake kinetics for fibrous structures with low-level total molded area.

### Example A

In a first instance fibrous structure comprising a 60/40 blend of polypropylene fibers and viscose fibers and having a basis weight of 58 gsm was hydromolded with an array of circular elements in a roughly hexagonal pattern as depicted in FIG. 26. The total molded area of this pattern relative to the total surface area of the fibrous structure is about 49%.

In a second instance, a similarly composed fibrous structure comprising a 60/40 blend of polypropylene fibers and viscose fibers and having a basis weight of 58 gsm was hydromolded with the same pattern, but wherein about 50% of the circular elements were removed, at random, from the pattern as depicted in FIG. 18. The total molded area of this pattern relative to the total surface area of the fibrous structure is about 25%.

In a third instance, a similarly composed fibrous structure comprising a 60/40 blend of polypropylene fibers and viscose fibers and having a basis weight of 58 gsm was not subject to hydromolding, thereby having a total molded area of about 0%.

Each of the fibrous structures described above was subject to the Fluid Uptake test method noted herein (below), with an impinging liquid whose composition is noted (below) as Example F. The fluid uptake kinetics for each of the described fibrous structures are given in Table 1.

TABLE 1

| % Molded Area | Fluid Uptake Kinetics (msec) |
|---|---|
| 0 | 0.57 |
| 25 | 0.49 |
| 49 | 0.59 |

### Example B

Similar to the example presented as Example A, a second series of fibrous structures comprising a 60/40 blend of pulp and Lyocell fibers and having a basis weight of 60 gsm were hydromolded with an array of circular elements in a roughly hexagonal pattern as depicted in FIGS. 26 and 18, with total molded area of this pattern relative to the total surface area of the fibrous structure is about 49% and about 25%, respectively, and compared with a similar fibrous structure without hydromolding, having a total molded area of about 0%.

Each of the fibrous structures was subject to the Fluid Uptake test method noted herein (below), with an impinging liquid whose composition is noted (below) as Example F. The fluid uptake kinetics for each of the described fibrous structures are given in Table 2.

TABLE 2

| % Molded Area | Fluid Uptake Kinetics (msec) |
|---|---|
| 0 | 0.57 |
| 25 | 0.39 |
| 49 | 0.44 |

### Example C

A fibrous structure comprising a 60/40 blend of polypropylene fibers and viscose fibers was hydromolded with the pattern depicted in FIG. 20. The total molded area of this pattern relative to the total surface area of the fibrous structure is about 29%, and this pattern includes the use of a “hollow” molded element, thereby exhibiting a high texture density relative to its low total molded area.

The fibrous structure was subject to the Fluid Uptake test method noted herein (below), with an impinging liquids whose composition is noted (below) as Example D and Example E. The fluid uptake kinetics for the fibrous structure with the impinging liquids of Example D and Example E are given in Tables 3 & 4, respectively.

TABLE 3

| % Molded | |
|---|---|
| Area | Fluid Uptake Kinetics (msec) |
| 0 | 0.88 |
| 29 | 0.77 |

TABLE 4

| % Molded | |
|---|---|
| Area | Fluid Uptake Kinetics (msec) |
| 0 | 0.55 |
| 29 | 0.53 |

### Example D

| | | Amount |
|---|---|---|
| Component | | (% by weight) |
| (1) | Disodium EDTA | 0.10 |
| (2) | Xanthan Gum | 0.18 |
| (3) | Abil Care 85 ™* | 0.45 |
| (4) | Sodium Dihydrogen Phosphate | 0.20 |
| | (Monohydrate) | |
| (5) | Benzyl Alcohol | 0.50 |
| (6) | PEG-40 Hydrogenated Castor Oil | 0.88 |
| (7) | Citric Acid | 0.05 |
| (8) | Iodopropynylbutylcarbamate | 0.009 |
| (9) | Hydroxymethylglycinate (50% aqueous) | 0.15 |
| (10) | Perfume | 0.05 |
| (11) | Purified Water | Balance |
| Total | | 100.00 |

*Abil Care 85 ™ comprises Bis-PEG/PPG-16/16 PEG/PPG Dimethicone Caprylic Capric triglyceride and is commercialized by Goldschmidt/Degussa, Goldschmidt AG, 45127 Essen, Germany www.goldschmidt.com.

### Example E

| | | Amount |
|---|---|---|
| Component | | (% by weight) |
| (1) | Disodium EDTA | 0.10 |
| (2) | Xanthan Gum | 0.18 |
| (3) | Abil Care 85 ™* | 0.10 |
| (4) | Trilaureth-4 Phosphate | 0.40 |
| (5) | Sodium Dihydrogen Phosphate | 0.18 |
| | (Monohydrate) | |
| (6) | Phenoxyethanol | 0.80 |
| (7) | PEG-40 Hydrogenated Castor Oil | 0.40 |
| (8) | Propylene Glycol | 1.50 |
| (9) | Methylparaben | 0.15 |
| (10) | Ethyl paraben | 0.05 |
| (11) | Propylparaben | 0.05 |
| (12) | Perfume | 0.05 |
| (13) | Purified Water | Balance |
| Total | | 100.00 |

*Abil Care 85 ™ comprises Bis-PEG/PPG-16/16 PEG/PPG Dimethicone Caprylic Capric triglyceride and is commercialized by Goldschmidt/Degussa, Goldschmidt AG, 45127 Essen, Germany www.goldschmidt.com.

### Example F

| | Amount |
|---|---|
| Component | (% by weight) |
| (1) Disodium EDTA | 0.10 |
| (2) Xanthan Gum | 0.18 |
| (3) Abil Care 85 ™* | 0.10 |
| (4) 1,2-Propyleneglycol | 1.50 |
| (5) Phenoxyethanol | 0.60 |
| (6) Methylparaben | 0.15 |
| (7) Propylparaben | 0.05 |
| (8) Ethylparaben | 0.05 |
| (9) Trilaureth-4 Phosphate | 0.40 |
| (10) PEG-40 Hydrogenated Castor Oil | 0.40 |
| (11) Perfume | 0.07 |
| (12) Purified water | Balance |
| Total | 100.00 |
| *Abil Care 85 ™ comprises Bis-PEG/PPG-16/16 PEG/PPG Dimethicone Caprylic Capric triglyceride and is commercialized by Goldschmidt/Degussa, Goldschmidt AG, 45127 Essen, Germany www.goldschmidt.com. The compositions of Examples G through N are further, non-limiting examples of compositions which may also be utilized as the impinging liquid or to impregnate the fibrous structure. The fibrous structure may be conveyed over a molding member comprising a molding pattern of any pattern | |
| #such as, but not limited to, those patterns illustrated in FIG. 3 through 24. | |

### Example G

| | Amount |
|---|---|
| Component | (% by weight) |
| (1) Disodium EDTA | 0.10 |
| (2) Arlatone-V 175 ™* | 0.80 |
| (3) Decylglycoside | 0.05 |
| (4) Cyclopentasiloxane Dimethiconol | 0.45 |
| (5) 1,2-Propyleneglycol | 1.50 |
| (6) Phenoxyethanol | 0.80 |
| (7) Methylparaben | 0.15 |
| (8) Propylparaben | 0.05 |
| (9) Ethylparaben | 0.05 |
| (10) PEG-40 Hydrogenated Castor Oil | 0.80 |
| (11) Perfume | 0.05 |
| (12) Purified water | Balance |
| Total | 100.00 |

*Arlatone-V 175 ™ comprises sucrose palmitate, glyceryl stearate, glyceryl stearate citrate, sucrose, mannan, xanthan gum and is commercialized by Uniqema GmbH&Co. KG 46429 Emmerich, Germany, www.uniqema.com.

### Example H

| | Amount |
|---|---|
| Component | (% by weight) |
| (1) Disodium EDTA | 0.10 |
| (2) Arlatone-V 175 ™* | 0.80 |
| (3) Abil Care 85 ™** | 0.45 |
| (4) Decylglycoside | 0.05 |
| (5) 1,2-Propyleneglycol | 1.50 |
| (6) Sodium benzoate | 0.20 |
| (7) Methylparaben | 0.15 |
| (8) Propylparaben | 0.05 |
| (9) Ethylparaben | 0.05 |
| (10) PEG-40 Hydrogenated Castor Oil | 0.80 |
| (11) Perfume | 0.05 |
| (12) Purified water | Balance |
| Total | 100.00 |

*Arlatone-V 175 ™ comprises sucrose palmitate, glyceryl stearate, glyceryl stearate citrate, sucrose, mannan, xanthan gum and is commercialized by Uniqema GmbH&Co. KG, 46429 Emmerich, Germany, www.uniqema.com.
**Abil Care 85 ™ comprises Bis-PEG/PPG-16/16 PEG/PPG Dimethicone Caprylic Capric triglyceride and is commercialized by Goldschmidt/Degussa, Goldschmidt AG, 45127 Essen, Germany www.goldschmidt.com.

### Example I

| | Amount |
|---|---|
| Component | (% by weight) |
| (1) Disodium EDTA | 0.10 |
| (2) Arlatone-V 175 ™* | 0.80 |
| (3) Cyclopentasiloxane Dimethiconol | 0.36 |
| (4) Glycerin | 0.067 |
| (5) Sodium trideceth carboxylate | 0.022 |
| (6) 1,-Propyleneglycol | 1.50 |
| (7) Phenoxyethanol | 0.60 |
| (8) Methylparaben | 0.15 |
| (9) Propylparaben | 0.05 |
| (10) Ethylparaben | 0.05 |
| (11) PEG-40 Hydrogenated Castor Oil | 0.80 |
| (12) Perfume | 0.05 |
| (13) Purified water | Balance |
| Total | 100.00 |

*Arlatone-V 175 ™ comprises sucrose palmitate, glyceryl stearate, glyceryl stearate citrate, sucrose, mannan, xanthan gum and is commercialized by Uniqema GmbH&Co. KG, 46429 Emmerich, Germany, www.uniqema.com.

### Example J

| | Amount |
|---|---|
| Component | (% by weight) |
| (1) Disodium EDTA | 0.10 |
| (2) Polysorbate 20 | 0.50 |
| (3) Simulgel NS ™* | 1.00 |
| (4) Abil Care 85 ™** | 1.00 |
| (5) Dimethicone | 1.00 |
| (6) C12-13 Alkylbenzoate | 0.50 |
| (7) 1,2-Propyleneglycol | 1.50 |
| (8) Sodium benzoate | 0.20 |
| (9) Methylparaben | 0.15 |
| (10) Propylparaben | 0.05 |
| (11) Ethylparaben | 0.05 |
| (12) PEG-40 Hydrogenated Castor Oil | 0.80 |
| (13) Perfume | 0.05 |
| (14) Purified water | Balance |
| Total | 100.00 |

*Simulgel NS ™ comprises Hydroxyethylacrylate/Sodium Acryloyldimethyltaurat copolymer&polysorbate60 and is commercialized by Seppic France, 75 Quai D′ Orsay, 75321 Paris Cedex 07, France, www.seppic.com.
**Abil Care 85 ™ comprises Bis-PEG/PPG-16/16 PEG/PPG Dimethicone Caprylic Capric triglyceride and is commercialized by Goldschmidt/Degussa, Goldschmidt AG, 45127 Essen, Germany www.goldschmidt.com.

### Example K

| | Amount |
|---|---|
| Component | (% by weight) |
| (1) Disodium EDTA | 0.10 |
| (2) Xanthan Gum | 0.18 |
| (3) Abil Care 85* | 0.10 |
| (4) Phenoxyethanol, Ethylhexyglycerine | 0.30 |
| (5) Benzyl Alcohol | 0.30 |
| (6) Sodium Benzoate | 0.12 |
| (7) PEG-40 Hydrogenated Castor Oil | 0.44 |
| (8) Trisodium Citrate | 0.33 |
| (9) Citric Acid | 0.53 |
| (10) Perfume | 0.05 |
| (11) Purified Water | Balance |
| Total | 100.00 |

*Abil Care 85 ™ comprises Bis-PEG/PPG-16/16 PEG/PPG Dimethicone Caprylic Capric triglyceride and is commercialized by Goldschmidt/Degussa, Goldschmidt AG, 45127 Essen, Germany www.goldschmidt.com.

### Example L

| | Amount |
|---|---|
| Component | (% by weight) |
| (1) Disodium EDTA | 0.10 |
| (2) Xanthan Gum | 0.18 |
| (3) Abil Care 85* | 0.45 |
| (4) Glycerine | 1.00 |
| (5) Phenoxyethanol | 0.30 |
| (6) Benzyl Alcohol | 0.30 |
| (7) Sodium Benzoate | 0.12 |
| (8) PEG-40 Hydrogenated Castor Oil | 0.44 |
| (9) Trisodium Citrate | 0.33 |
| (10) Citric Acid | 0.53 |
| (11) Perfume | 0.05 |
| (12) Purified Water | Balance |
| Total | 100.00 |

*Abil Care 85 ™ comprises Bis-PEG/PPG-16/16 PEG/PPG Dimethicone Caprylic Capric triglyceride and is commercialized by Goldschmidt/Degussa, Goldschmidt AG, 45127 Essen, Germany www.goldschmidt.com.

### Example M

| | Amount |
|---|---|
| Component | (% by weight) |
| Disodium EDTA | 0.10 |
| Xanthan Gum | 0.10 |
| Abil Care 85* | 0.10 |
| Phenoxyethanol | 0.30 |
| Benzyl Alcohol | 0.30 |
| Sodium Benzoate | 0.12 |
| PEG-40 Hydrogenated Castor Oil | 0.22 |
| Trisodium Citrate | 0.33 |
| Citric Acid | 0.53 |
| Perfume | 0.05 |
| Purified Water | Balance |
| Total | 100.00 |

*Abil Care 85 ™ comprises Bis-PEG/PPG-16/16 PEG/PPG Dimethicone Caprylic Capric triglyceride and is commercialized by Goldschmidt/Degussa, Goldschmidt AG, 45127 Essen, Germany www.goldschmidt.com.

### Example N

| | Amount |
|---|---|
| Component | (% by weight) |
| Disodium EDTA | 0.10 |
| Xanthan Gum | 0.18 |
| Abil Care 85* | 0.10 |
| Glycerine | 1.00 |
| Phenoxyethanol, Ethylhexyglycerine | 0.30 |
| Benzyl Alcohol | 0.30 |
| Sodium Benzoate | 0.12 |
| PEG-40 Hydrogenated Castor Oil | 0.44 |
| Trisodium Citrate | 0.33 |
| Citric Acid | 0.53 |
| Chamomille Extract | 0.003 |
| Perfume | 0.05 |
| Purified Water | Balance |
| Total | 100.00 |

*Abil Care 85 ™ comprises Bis-PEG/PPG-16/16 PEG/PPG Dimethicone Caprylic Capric triglyceride and is commercialized by Goldschmidt/Degussa, Goldschmidt AG, 45127 Essen, Germany www.goldschmidt.com.

Fluid Uptake Test Method

Fluid uptake measurements are made on a TRI/Upkin™ (TRI/Princeton Inc. of Princeton, N.J.). The TRI/Upkin measurement includes a sample of a fibrous structure or substrate and a liquid.

Sample Preparation

Sample Preparation—Fibrous Structure or Substrate:

The fibrous structure or substrate is cut to a 50 mm×50 mm square using a vendor provided template. The cut piece of the fibrous structure or substrate is then placed on top of a perforated plate in the TRI-Upkin equipment. The cover plate is placed over the fibrous structure or substrate sample.

Sample Preparation—Liquid:

Any impinging liquid can be used in the TRI-Upkin measurement. Examples of impinging liquids may be found in Examples D through N. The impinging liquid is loaded into a reservoir below the perforated plate (adjacent to the fibrous structure or substrate sample), and loaded into the TRI-Upkin equipment concurrent with the fibrous structure or substrate sample.

Procedure

As used in this application, determining the fluid uptake comprises recording the location of the fluid front as it advances throughout the fibrous network over time.

In the measurement, an automated motor brings the sample in contact with the liquid. As the liquid is drawn into the fibrous structure or substrate by capillary forces a sensor measures the average position of the moving liquid front in the sample every millisecond. Simultaneously, another sensor measures the contraction or expansion of the fibrous structure or substrate while it absorbs liquid. The data acquisition system simultaneously records the position of the moving liquid front in the sample's pores and the sample's thickness. When the sample reaches saturation and there is no further change in thickness, the computer stops the data acquisition, activates the motor that raises the sample holder, and ends the experiment.

The fluid uptake measurement is taken as the time required for the fluid front to penetrate 35% of the transplanar thickness of the fibrous structure or substrate sample.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.

All documents cited in the Detailed Description of the invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and the scope of the invention. It is therefore intended to cover in the appended claims all such changes and modifications that are within the scope of this invention.

What is claimed is:

1. A molded fibrous structure comprising from about 5 to about 49% molded area wherein said molded area comprises at least one molded element.

2. The fibrous structure of claim 1 comprising from about 5 to about 45% molded area.

3. The fibrous structure of claim 1 comprising from about 15 to about 35% molded area.

4. The fibrous structure of claim 1 comprising synthetic fibers, natural fibers or combinations thereof.

5. The fibrous structure of claim 4 wherein said synthetic fibers may comprise materials selected from the group consisting of polyesters, polyolefins, polypropylenes, polyethylenes, polyethers, polyamides, polyesteramides, polyvinylalcohols, polyhydroxyalkanoates, polysaccharides and combinations thereof.

6. The fibrous structures of claim 4 wherein said natural fibers comprise materials selected from the group consisting of cellulose, starch, wood pulp, typical northern softwood Kraft, typical southern softwood Kraft, typical CTMP, typical deinked, corn pulp, acacia, eucalyptus, aspen, reed pulp, birch, maple, radiata pine, albardine, esparto, wheat, rice, corn, sugar cane, papyrus, jute, reed, sabia, raphia, bamboo, sidal, kenaf, abaca, sunn, rayon, lyocell, cotton, hemp, flax, ramie, down, feathers, silk, and combinations thereof.

7. The fibrous structure of claim 1 wherein said molded element is hollow.

8. The fibrous structure of claim 1 wherein said molded element is selected from the group consisting of circles, squares, rectangles, ovals, ellipses, irregular circles, swirls, curly cues, cross hatches, pebbles, lined circles, linked irregular circles, half circles, wavy lines, bubble lines, puzzles, leaves, outlined leaves, plates, connected circles, changing curves, dots, honeycombs, and combinations thereof.

9. The fibrous structure of claim 1 wherein said molded element is selected from the group consisting of logos, indicia, trademarks, geometric patterns, surface images, and combinations thereof.

10. The fibrous structure of claim 1 wherein said molded element is arranged in a repeating pattern on said fibrous structure.

11. The fibrous structure of claim 1 wherein said fibrous structure comprises at least two molded element wherein one of said at least two molded elements is smaller than the other of said at least two molded elements.

12. The fibrous structure of claim 11 wherein said smaller molded element comprises a radius unit.

13. The fibrous structure of claim 12 wherein said smaller molded element is disposed within 4 radius units of the other of said at least two molded elements.

14. The fibrous structure of claim 11 wherein said first and said second molded elements provide a high texture impression.

15. A substrate comprising said fibrous structure of claim 1.

16. The substrate of claim 15 further comprising a composition that is suitable for a purpose selected from the group consisting of cleansing, skin soothing, moisturizing, exfoliating, and combinations thereof.
